(19) United States
(12) Patent Application Publication
(10) Pub. No.: US 2026/0294613 A1
(43) Pub. Date: Oct. 1, 2026

(54) ENDOPROSTHESIS FOR HEPATICOGASTROSTOMY USE

(71) Applicant: BOSTON SCIENTIFIC SCIMED, INC., Maple Grove, MN (US)

(72) Inventor: MARTYN G. FOLAN, Loughrea (IE)

(73) Assignee: BOSTON SCIENTIFIC SCIMED, INC., Maple Grove, MN (US)

(21) Appl. No.: 19/629,688

(22) Filed: Mar. 26, 2026

Related U.S. Application Data
(60) Provisional application No. 63/778,725, filed on Mar. 27, 2025.

(51) Int. Cl. A61F 2/04

(52) U.S. Cl. CPC A61F 2/04; A61F 2002/045; A61F 2220/0008; A61F 2230/0039; A61F 2250/0039

(57) ABSTRACT

An endoprosthesis includes a body portion and a flared portion extending between the body portion and a first end. The body and flared portions are formed from a single wire. The body portion includes an undulating arrangement of first and second struts defining peaks and valleys. The body portion includes a first circumferential end segment at a first body end oriented perpendicular to a central axis, a second circumferential end segment at a second body end oriented perpendicular to the central axis, and a plurality of circumferential segments extending helically around the central axis between the first and second end segments. The flared portion includes a plurality of knitted circumferential segments extending circumferentially around the central axis from the body portion to the first end. Centroids of the first and second struts within the first and second circumferential end segments lie within planes oriented perpendicular to the central axis.

### CROSS REFERENCE TO RELATED APPLICATIONS

The present application claims the benefit of US Provisional Patent Application Serial No. 63/778,725, filed Mar. 27, 2025, the disclosure of which is incorporated herein by reference.

### TECHNICAL FIELD

The present disclosure pertains to medical devices, methods for manufacturing medical devices, and uses thereof. More particularly, the present disclosure pertains to an endoprosthesis or stent for implantation in a body lumen, and associated methods.

### BACKGROUND

An endoprosthesis may be used in the treatment of body lumens. One type of endoprosthesis used in the repair and/or treatment of diseases in various body lumens is a stent. A stent is a generally longitudinal tubular device formed of biocompatible material which is useful to open and support various lumens in the body. For example, stents may be used in the vascular system, urogenital tract, gastrointestinal tract, esophageal tract, tracheal/bronchial tubes, the biliary tract, etc. as well as in a variety of other applications in the body.

In some instances, it may be desirable to design an endoprosthesis to include sufficient flexibility and conformability to the body lumen, while maintaining sufficient radial force to open the body lumen at the treatment site and/or prevent migration of the endoprosthesis within the body lumen. In some instances, it may be desirable to reduce or limit foreshortening. In some instances, it may be desirable to reduce or limit migration. In some instances, different endoprosthesis configurations may provide different deliverability, flexibility, conformability, radial force/strength, and/or anchoring/migration characteristics.

Various medical procedures involve the temporary or permanent joining of non-connected anatomical structures. Some examples include a hepaticogastrostomy (HG) involving joining the hepatic duct and the stomach to drain the bile duct, a gastrojejeneum (GJ) bypass procedure to create an anastomosis between the small intestine and stomach wall, and stomas to create an artificial opening into the large intestine or other region of the digestive tract. In these medical procedures, peristalsis and gross organ movement in one or both of the anatomical structures being connected may create difficulties in using stents to join the structures due to stent migration or other complications.

Of the known medical devices and methods, each has certain advantages and disadvantages. There is an ongoing need to provide alternative medical devices as well as alternative methods for manufacturing and using medical devices.

### SUMMARY

In one example, an endoprosthesis extending axially along a central longitudinal axis between a first end and a second end may comprise a body portion and a flared portion extending between the body portion and the first end. The body portion and the flared portion may be formed from a single wire extending from the first end to the second end. The body portion may comprise an undulating arrangement of first struts and second struts defining peaks and valleys, wherein the body portion comprises a first circumferential end segment extending circumferentially around the central longitudinal axis at a first body end, wherein the first circumferential end segment is oriented generally perpendicular to the central longitudinal axis, a second circumferential end segment extending circumferentially around the central longitudinal axis at a second body end, wherein the second circumferential end segment is oriented generally perpendicular to the central longitudinal axis, and a plurality of circumferential segments extending helically around the central longitudinal axis between the first circumferential end segment and the second circumferential end segment. The flared portion may comprise a plurality of knitted circumferential segments extending circumferentially around the central longitudinal axis from the body portion to the first end.

In addition, or alternatively, to any example disclosed herein, the plurality of knitted circumferential segments comprises a first knitted circumferential segment disposed immediately adjacent the first circumferential end segment.

In addition, or alternatively, to any example disclosed herein, the single wire comprises a transition region extending from the first circumferential end segment to the first knitted circumferential segment.

In addition, or alternatively, to any example disclosed herein, the transition region extends in a generally axial direction without crossing over any portion of the single wire disposed within the first circumferential end segment.

In addition, or alternatively, to any example disclosed herein, the transition region extends circumferentially and is interwoven through the first circumferential end segment.

In addition, or alternatively, to any example disclosed herein, the first knitted circumferential segment of the plurality of knitted circumferential segments is interwoven with the first circumferential end segment.

In addition, or alternatively, to any example disclosed herein, the first knitted circumferential segment of the plurality of knitted circumferential segments is interwoven with the transition region of the single wire.

In addition, or alternatively, to any example disclosed herein, the plurality of circumferential segments is oriented at an oblique angle relative to the central longitudinal axis, as viewed in a flat pattern configuration.

In addition, or alternatively, to any example disclosed herein, the endoprosthesis may comprise a polymeric covering fixedly attached to the body portion and the flared portion.

In addition, or alternatively, to any example disclosed herein, at least one circumferential segment of the plurality of circumferential segments disposed within the body portion comprises an anti-migration element projecting radially outward from the at least one circumferential segment of the plurality of circumferential segments disposed within the body portion, wherein the anti-migration element is formed from the single wire.

In addition, or alternatively, to any example disclosed herein, the body portion is configured to be disposed within a first body lumen and the flared portion is configured to project into an interior space formed within a second body lumen such that the first end is spaced apart from a wall of the second body lumen.

In addition, or alternatively, to any example disclosed herein, the first body lumen is a duct of a biliary system of a patient and the second body lumen is a stomach of the patient.

In addition, or alternatively, to any example disclosed herein, the endoprosthesis may comprise a second flared portion formed from the single wire and extending between the body portion and the second end.

In addition, or alternatively, to any example disclosed herein, the second flared portion comprises a second plurality of knitted circumferential segments extending circumferentially around the central longitudinal axis from the body portion to the second end.

In addition, or alternatively, to any example disclosed herein, and in a second example, an endoprosthesis extending axially along a central longitudinal axis between a first end and a second end may comprise a body portion and a flared portion extending between the body portion and the first end. The body portion and the flared portion may be formed from a single wire extending from the first end to the second end. The body portion may comprise an undulating arrangement of first struts and second struts defining peaks and valleys, wherein the body portion comprises a first circumferential end segment extending circumferentially around the central longitudinal axis at a first body end, wherein centroids of the first struts and the second struts disposed within the first circumferential end segment lie within a first plane oriented generally perpendicular to the central longitudinal axis, a second circumferential end segment extending circumferentially around the central longitudinal axis at a second body end, wherein centroids of the first struts and the second struts disposed within the second circumferential end segment lie within a second plane oriented generally perpendicular to the central longitudinal axis, and a plurality of circumferential segments extending helically around the central longitudinal axis between the first circumferential end segment and the second circumferential end segment. The flared portion may comprise a plurality of knitted circumferential segments extending circumferentially around the central longitudinal axis from the body portion to the first end.

In addition, or alternatively, to any example disclosed herein, centroids of the first struts and the second struts disposed within the plurality of circumferential segments are non-coplanar.

In addition, or alternatively, to any example disclosed herein, centroids of the first struts and the second struts disposed within the plurality of circumferential segments are arranged helically around the central longitudinal axis.

In addition, or alternatively, to any example disclosed herein, and in a third example, an endoprosthesis having a first end and a second end may comprise a body portion extending axially along a central longitudinal axis from the second end toward the first end, a first end portion extending axially along a central longitudinal axis from the first end toward the second end, and a flanged portion extending between the body portion and the first end portion. The body portion, the first end portion, and the flanged portion may be formed from a single wire extending from the first end to the second end. The body portion may comprise an undulating arrangement of first struts and second struts defining peaks and valleys, wherein the body portion comprises a first circumferential end segment extending circumferentially around the central longitudinal axis at a first body end, wherein the first circumferential end segment is oriented generally perpendicular to the central longitudinal axis, a second circumferential end segment extending circumferentially around the central longitudinal axis at a second body end, wherein the second circumferential end segment is oriented generally perpendicular to the central longitudinal axis, and a plurality of circumferential segments extending helically around the central longitudinal axis between the first circumferential end segment and the second circumferential end segment. The flanged portion may comprise a plurality of knitted circumferential segments extending circumferentially around the central longitudinal axis from the body portion to the first end portion. The plurality of knitted circumferential segments may extend circumferentially around the central longitudinal axis within the first end portion from the flanged portion to the first end.

In addition, or alternatively, to any example disclosed herein, the endoprosthesis may comprise a polymeric covering fixedly attached to the body portion, the first end portion, and the flanged portion.

In addition, or alternatively, to any example disclosed herein, the body portion is configured to be disposed within a first body lumen and the first end portion is configured to project into an interior space formed within a second body lumen such that the first end is spaced apart from a wall of the second body lumen. The body portion is configured to span a gap between the first body lumen and the second body lumen.

The above summary of some embodiments, aspects, and/or examples is not intended to describe each disclosed embodiment or every implementation of the present disclosure. The figures and detailed description which follow more particularly exemplify these embodiments .

### BRIEF DESCRIPTION OF THE DRAWINGS

The disclosure may be more completely understood in consideration of the following detailed description in connection with the accompanying drawings, in which:

FIG. 1 schematically illustrates selected portions of a patient’s anatomy;

FIGS. 2-3 are flat pattern views illustrating selected aspects of an endoprosthesis in accordance with the disclosure;

FIG. 4 is a side view illustrating selected aspects of an endoprosthesis in accordance with the disclosure;

FIG. 4A is a detail view of a portion of FIG. 4;

FIG. 5 is a side view illustrating selected aspects of an endoprosthesis in accordance with the disclosure;

FIG. 5A is a detail view of a portion of FIG. 5;

FIG. 5B is a detail view of a portion of FIG. 5;

FIG. 6 is a side view illustrating selected aspects of an endoprosthesis in accordance with the disclosure;

FIG. 6A is a detail view of a portion of FIG. 6;

FIG. 7 is a partial cutaway view illustrating selected aspects of an endoprosthesis in accordance with the disclosure disposed within first and second body lumens of a patient; and

FIGS. 8-12 are side views illustrating selected aspects of an endoprosthesis in accordance with the disclosure.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

In some instances, it may be desirable to provide an endoprosthesis, or stent, that can deliver luminal patency within the pancreaticobiliary tree of a patient. The relatively narrow biliary tract ducts consist of a series of bifurcations linking the liver, gallbladder, and pancreas via the papilla to the duodenal space for the transportation of bile and related enzymic substances for many metabolic functions but most commonly the body’s ability to digest and absorb fats and vitamins D and K. Endoscopic retrograde cholangiopancreatography (ERCP) may be used to diagnose and treat conditions of the bile ducts, including, for example, gallstones, inflammatory strictures, leaks (e.g., from trauma, surgery, etc.), and cancer. Blockage of the biliary duct may occur in many of the disorders of the biliary system, including the disorders of the liver, such as, primary schlerosing cholangitis, stone formation, scarring in the duct, etc. Draining blocked fluids from the biliary system may be used to treat the disorders. However, ERCP may not always be an option or may be unsuccessful in difficult anatomies or challenging disease states. Hepaticogastrostomy (HG) may be used in cases where ERCP is not an option or fails. Other procedures, such as, but not limited to, choledochoduodenostomy (CDS) may be used to directly connect the common bile duct to the duodenum.

Hepaticogastrostomy Stenting (HGS) is a procedure that targets a hepatic duct within the liver directly from the stomach and places an endoprosthesis or stent creating an artificial pathway (bridging the peritoneal cavity) to facilitate ongoing internal biliary drainage. The HGS procedure is completed using an endoscopic ultrasound scope or other endoscope advanced through the esophagus that allows the endoscopist to “see” through the wall of the stomach and into the liver prior to gaining access. The endoscopist may position the scope with an appropriate clear trajectory to the hepatic duct. Guidewire access to the hepatic duct is then made using a needle followed by enlargement of the tract using a balloon and/or cystotome. Alternatively, access may be gained using a specialized all in one device, such as the Axios™ ‘Hot’ delivery device from Boston Scientific. The guidewire is advanced from the stomach into the duct. An endoprosthesis or stent is advanced and deployed to create and maintain a bridge between the hepatic duct and an interior of the stomach thus facilitating ongoing internal biliary drainage.

An additional factor endoscopists balance when positioning the endoscope for this procedure is also the proximal positioning of the endoprosthesis or stent within the stomach to minimize food impaction/migration into the lumen of the endoprosthesis or stent. Migration of the endoprosthesis or stent out of the stomach may be one of the highest severity risks with HGS. Migration of the endoprosthesis or stent out of the stomach can result in leakage and tissue damage (e.g., biliary peritonitis). Additionally, the migrated stent is free to abrade the outer gastric wall and other organs or vessels in the vicinity.

The hepatic duct and the stomach are spaced apart. The distance between the organs may vary and requires a relatively long stent. As the stomach muscles contract during digestion, the distance between the wall of the stomach relative to the hepatic duct varies, from a relatively small distance when the stomach is relaxed to a greater distance when the stomach is contracted. In addition to the wall of the stomach flexing, the stomach undergoes peristalsis during digestion. This relative motility of the stomach is understood to be complex, in three dimensions rather than in an exclusively linear manner. The distance between the target organs to be joined, as well as the relative movement of at least one of the organs, may increase the chance of stent migration.

An additional concern with traditional braided stents used in this indication is foreshortening which may cause deployment accuracy issues. The present disclosure is directed towards endoprostheses or stents for use in HGS which inhibit migration and/or inhibit or limit foreshortening.

While the present disclosure is described with respect to an HGS procedure, the devices, systems, and/or methods described herein may be used in stents, endoluminal implants, or transluminal implants where the proximal end terminates in the upper GI tract. This may include devices placed during a CDS procedure or during a successful ERCP procedure, among others. Further, while the present disclosure is described with respect to the pancreaticobiliary ductal system, the devices, systems, and/or methods described herein may be used in stents, endoluminal implants, or transluminal implants positioned in other parts of the body, such as, but not limited to, bodily tissue, bodily organs, vascular lumens, non-vascular lumens and combinations thereof, such as, but not limited to, in the coronary or peripheral vasculature, trachea, bronchi, colon, small intestine, esophagus, biliary tract, urinary tract, prostate, brain, stomach, and the like.

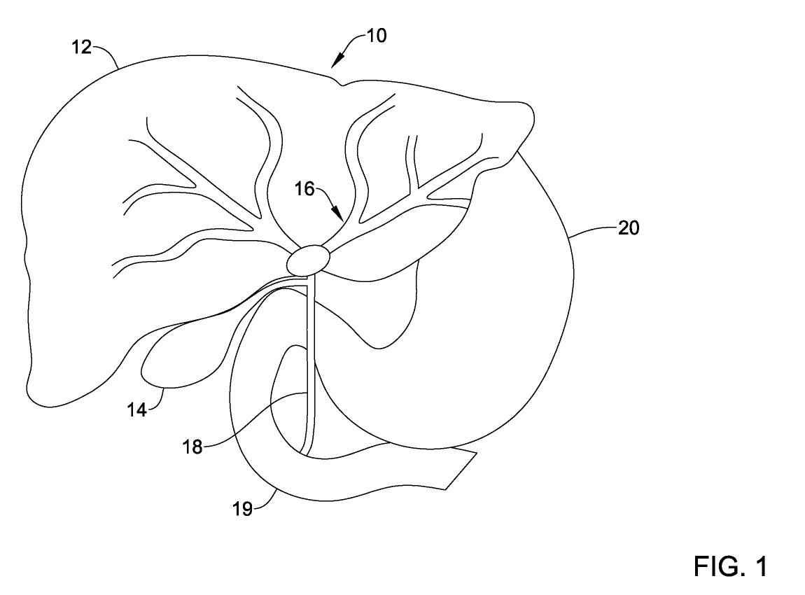

FIG. 1 schematically illustrates selected portions of a patient’s anatomy. The biliary system 10 may include, among other things given anatomical variations, the liver 12, the gall bladder 14, hepatic ducts 16, and the common bile duct 18. The common bile duct 18 connects to the duodenum 19 extending downstream from the stomach 20. In some cases, the common bile duct 18, or other lumens of the biliary system 10, may become blocked (partially or fully). In order to facilitate and/or restore biliary drainage, an HGS procedure may be performed to fluidly couple the hepatic ducts 16 to the stomach 20. As discussed herein, the stomach 20 and the liver 12 may move relative to each other within the peritoneal space during digestion and/or at other times as there is no direct attachment or connection between these two organs.

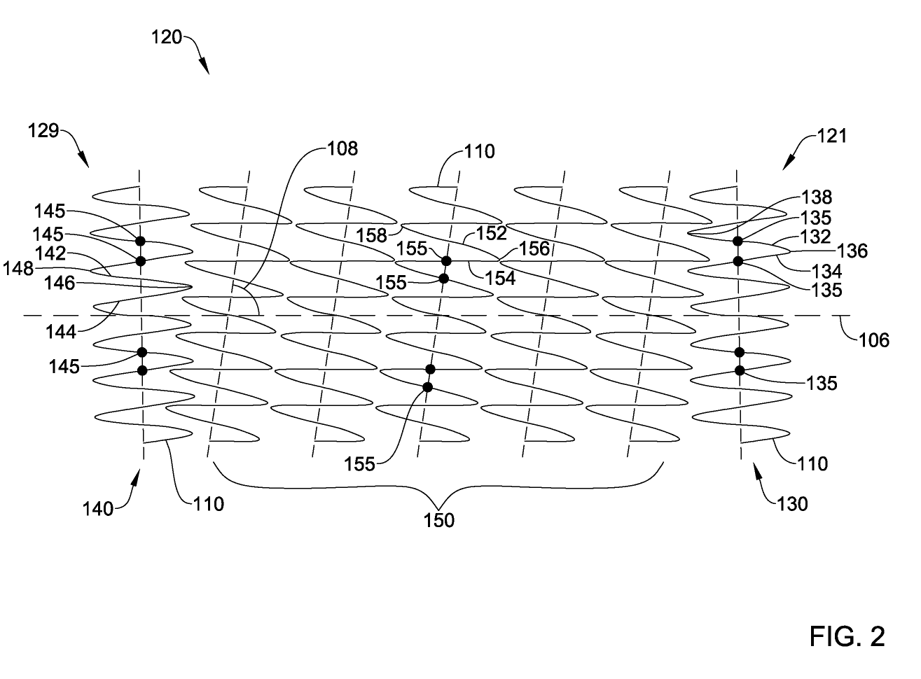

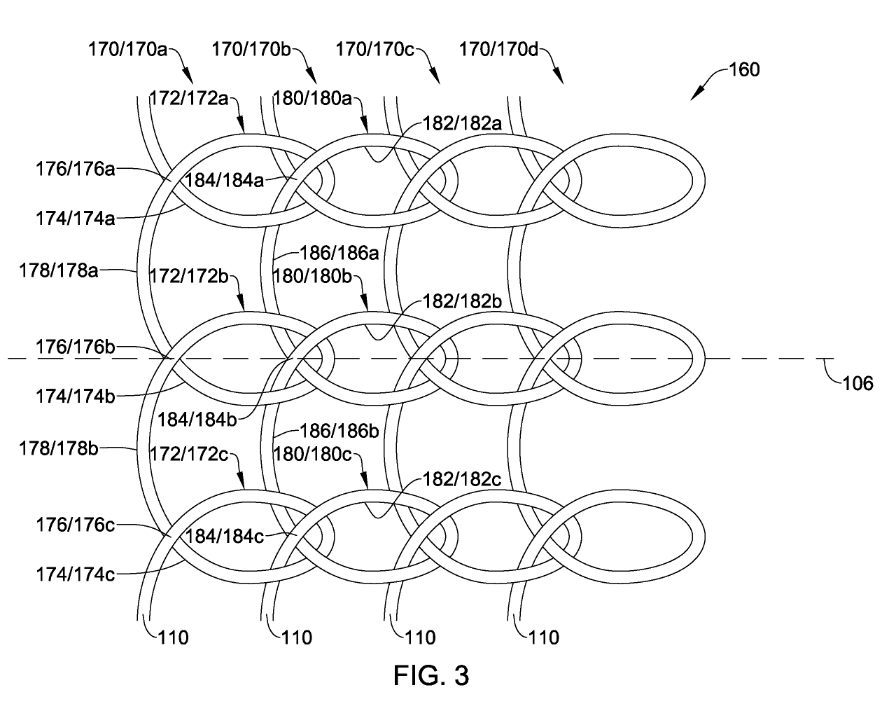

FIGS. 2-3 schematically illustrate selected aspects related to the construction of an endoprosthesis 100, or a stent, in flat pattern views. This may be referred to herein as a flat pattern configuration.

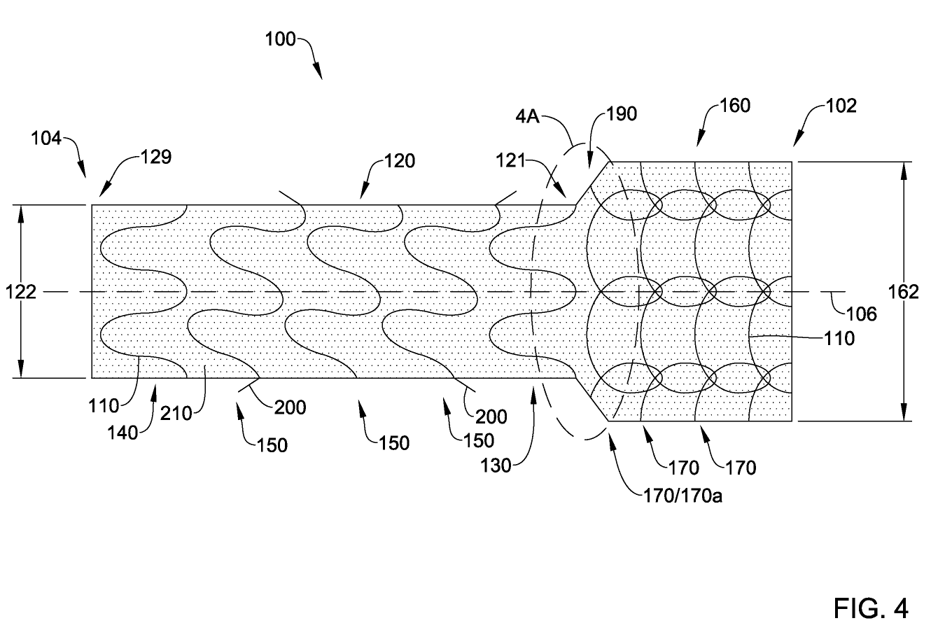

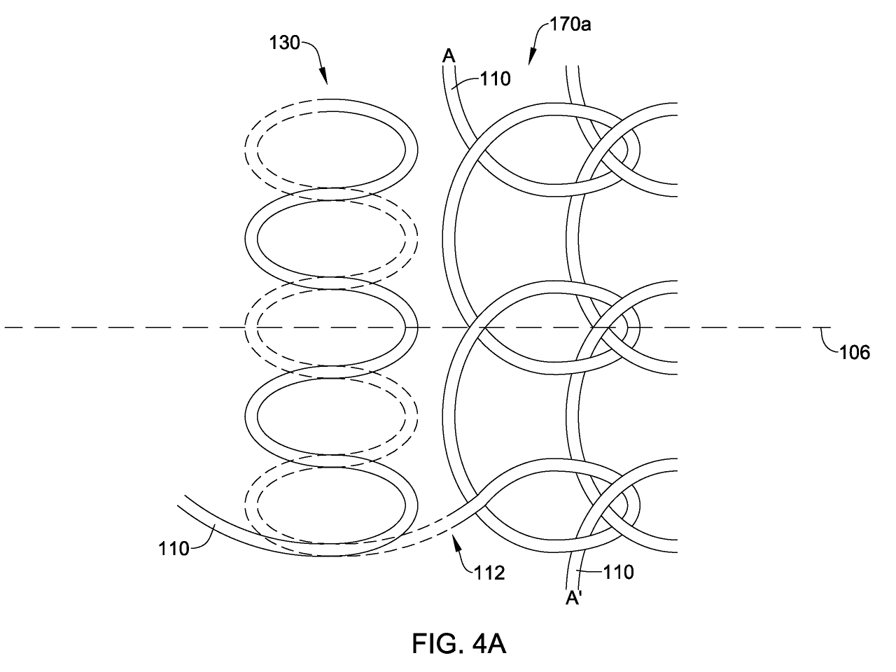

The endoprosthesis 100 may have a first end 102 (e.g., FIG. 4) and a second end 104 (e.g., FIG. 4). The endoprosthesis 100 may extend axially along a central longitudinal axis 106. In some embodiments, the endoprosthesis 100 may be formed from a single wire 110 extending from the first end 102 to the second end 104. In some embodiments, the first end 102 and the second end 104 may define a length of the endoprosthesis 100 and/or an overall length of the endoprosthesis 100. In at least some embodiments, the endoprosthesis 100 may be tubular. The endoprosthesis 100 may define a lumen extending therethrough from the first end 102 to the second end 104. The single wire 110 may extend circumferentially around the central longitudinal axis 106 and/or the single wire 110 may encircle the central longitudinal axis 106. The single wire 110 is and/or consists of only one wire.

Some suitable but non-limiting examples of materials for the endoprosthesis 100 and/or the single wire 110, such as metallic materials, composite materials, shape memory materials, combinations thereof, etc., are discussed below. In one non-limiting example, the endoprosthesis 100 and/or the single wire 110 may be formed from nickel-titanium alloy (e.g., nitinol). Other configurations and/or materials are also contemplated.

In some embodiments, the endoprosthesis 100 may comprise a body portion 120 comprising an undulating arrangement of first struts and second struts defining peaks and valleys, as seen in FIG. 2 and in FIG. 4. In some embodiments, the body portion 120 may extend from a first body end 121 to a second body end 129.

In some embodiments, the body portion 120 may comprise and/or the single wire 110 may form a first circumferential end segment 130 extending circumferentially around the central longitudinal axis 106 at and/or proximate the first body end 121. The first circumferential end segment 130 may comprise an undulating arrangement (e.g., a sinusoidal arrangement) of first struts 132 and second struts 134 defining peaks 136 and valleys 138. In at least some embodiments, the first struts 132 of first circumferential end segment 130 have a first length defined by the peaks 136 and the valleys 138 and the second struts 134 of first circumferential end segment 130 have a second length defined by the peaks 136 and the valleys 138, wherein the first length of the first struts 132 is equal to the second length of the second struts 134. In some embodiments, the first length of the first struts 132 of first circumferential end segment 130 and the second length of the second struts 134 of first circumferential end segment 130 may be uniform and/or constant within the first circumferential end segment 130. In some alternative embodiments, the first length of the first struts 132 of first circumferential end segment 130 and the second length of the second struts 134 of first circumferential end segment 130 may vary within the first circumferential end segment 130.

In some embodiments, the first length of the first struts 132 of first circumferential end segment 130 and/or the second length of the second struts 134 of first circumferential end segment 130 may be between about 1 millimeter and about 15 millimeters. In some embodiments, the first length of the first struts 132 of first circumferential end segment 130 and/or the second length of the second struts 134 of first circumferential end segment 130 may be between about 3 millimeters and about 12 millimeters. In some embodiments, the first length of the first struts 132 of first circumferential end segment 130 and/or the second length of the second struts 134 of first circumferential end segment 130 may be between about 5 millimeters and about 10 millimeters. Other configurations are also contemplated.

In at least some embodiments, the first circumferential end segment 130 may be oriented generally perpendicular to the central longitudinal axis 106. In some embodiments, each strut (e.g., the first struts 132, the second struts 134) of the first circumferential end segment 130 may comprise and/or define a centroid 135 disposed equidistantly between the peaks 136 and the valleys 138. The centroid(s) 135 of each strut (e.g., the first struts 132, the second struts 134) disposed within the first circumferential end segment 130 may lie in a first plane oriented perpendicular to the central longitudinal axis 106. In some embodiments, the peaks 136 of the first circumferential end segment 130 may touch and/or lie in a plane oriented perpendicular to the central longitudinal axis 106. In some embodiments, the valleys 138 of the first circumferential end segment 130 may touch and/or lie in a plane oriented perpendicular to the central longitudinal axis 106.

In some embodiments, the body portion 120 may comprise and/or the single wire 110 may form a second circumferential end segment 140 extending circumferentially around the central longitudinal axis 106 at and/or proximate the second body end 129. The second circumferential end segment 140 may comprise an undulating arrangement (e.g., a sinusoidal arrangement) of first struts 142 and second struts 144 defining peaks 146 and valleys 148. In at least some embodiments, the first struts 142 of the second circumferential end segment 140 have a first length defined by the peaks 146 and the valleys 148 and the second struts 144 of the second circumferential end segment 140 have a second length defined by the peaks 146 and the valleys 148, wherein the first length of the first struts 142 of the second circumferential end segment 140 is equal to the second length of the second struts 144 of the second circumferential end segment 140. In some embodiments, the first length of the first struts 142 of the second circumferential end segment 140 and the second length of the second struts 144 of the second circumferential end segment 140 may be uniform and/or constant within the second circumferential end segment 140. In some alternative embodiments, the first length of the first struts 142 of the second circumferential end segment 140 and the second length of the second struts 144 of the second circumferential end segment 140 may vary within the second circumferential end segment 140.

In some embodiments, the first length of the first struts 142 of the second circumferential end segment 140 and/or the second length of the second struts 144 of the second circumferential end segment 140 may be between about 1 millimeter and about 15 millimeters. In some embodiments, the first length of the first struts 142 of the second circumferential end segment 140 and/or the second length of the second struts 144 of the second circumferential end segment 140 may be between about 3 millimeters and about 12 millimeters. In some embodiments, the first length of the first struts 142 of the second circumferential end segment 140 and/or the second length of the second struts 144 of the second circumferential end segment 140 may be between about 5 millimeters and about 10 millimeters. Other configurations are also contemplated.

In at least some embodiments, the second circumferential end segment 140 may be oriented generally perpendicular to the central longitudinal axis 106. In some embodiments, each strut (e.g., the first struts 142, the second struts 144) of the second circumferential end segment 140 may comprise and/or define a centroid 145 disposed equidistantly between the peaks 146 and the valleys 148. The centroid(s) 145 of each strut (e.g., the first struts 142, the second struts 144) disposed within the second circumferential end segment 140 may lie in a second plane oriented perpendicular to the central longitudinal axis 106. In some embodiments, the peaks 146 of the second circumferential end segment 140 may touch and/or lie in a plane oriented perpendicular to the central longitudinal axis 106. In some embodiments, the valleys 148 of the second circumferential end segment 140 may touch and/or lie in a plane oriented perpendicular to the central longitudinal axis 106.

In some embodiments, the body portion 120 may comprise and/or the single wire 110 may form a plurality of circumferential segments 150 extending helically around the central longitudinal axis 106 between the first circumferential end segment 130 and the second circumferential end segment 140. In some embodiments, the plurality of circumferential segments 150 may comprise two circumferential segments, three circumferential segments, four circumferential segments, etc. up to a desired number of circumferential segments that combine with the first circumferential end segment 130 and the second circumferential end segment 140 to produce a desired overall length for the body portion 120 and/or the endoprosthesis 100.

In some embodiments, each circumferential segment of the plurality of circumferential segments 150 may comprise an undulating arrangement (e.g., a sinusoidal arrangement) of first struts 152 and second struts 154 defining peaks 156 and valleys 158. In at least some embodiments, the first struts 152 of each circumferential segment of the plurality of circumferential segments 150 have a first length defined by the peaks 156 and the valleys 158 and the second struts 154 of each circumferential segment of the plurality of circumferential segments 150 have a second length defined by the peaks 156 and the valleys 158, wherein the first length of the first struts 152 of each circumferential segment of the plurality of circumferential segments 150 is equal to the second length of the second struts 154 of each circumferential segment of the plurality of circumferential segments 150. In some embodiments, the first length of the first struts 152 of each circumferential segment of the plurality of circumferential segments 150 and the second length of the second struts 154 of each circumferential segment of the plurality of circumferential segments 150 may be uniform and/or constant within each circumferential segment of the plurality of circumferential segments 150. In some alternative embodiments, the first length of the first struts 152 of each circumferential segment of the plurality of circumferential segments 150 and the second length of the second struts 154 of each circumferential segment of the plurality of circumferential segments 150 may vary within each circumferential segment of the plurality of circumferential segments 150.

In some embodiments, the first length of the first struts 152 of each circumferential segment of the plurality of circumferential segments 150 and/or the second length of the second struts 154 of each circumferential segment of the plurality of circumferential segments 150 may be between about 1 millimeter and about 15 millimeters. In some embodiments, the first length of the first struts 152 of each circumferential segment of the plurality of circumferential segments 150 and/or the second length of the second struts 154 of each circumferential segment of the plurality of circumferential segments 150 may be between about 3 millimeters and about 12 millimeters. In some embodiments, the first length of the first struts 152 of each circumferential segment of the plurality of circumferential segments 150 and/or the second length of the second struts 154 of each circumferential segment of the plurality of circumferential segments 150 may be between about 5 millimeters and about 10 millimeters. Other configurations are also contemplated.

In some embodiments, the plurality of circumferential segments 150 may extend helically around the central longitudinal axis 106 from the first circumferential end segment 130 to the second circumferential end segment 140. In some embodiments, each strut (e.g., the first struts 152, the second struts 154) of each circumferential segment of the plurality of circumferential segments 150 may comprise and/or define a centroid 155 disposed equidistantly between the peaks 156 and the valleys 158.

In some embodiments, the centroid(s) 155 of each strut (e.g., the first struts 152, the second struts 154) of each circumferential segment of the plurality of circumferential segments 150 may extend and/or may be arranged helically around the central longitudinal axis 106 from the first circumferential end segment 130 to the second circumferential end segment 140. In some embodiments, the peaks 156 may extend and/or may be arranged helically around the central longitudinal axis 106 from the first circumferential end segment 130 to the second circumferential end segment 140. In some embodiments, the valleys 158 may extend and/or may be arranged helically around the central longitudinal axis 106 from the first circumferential end segment 130 to the second circumferential end segment 140. In at least some embodiments, the centroid(s) 155 of each strut (e.g., the first struts 152 and the second struts 154) of each circumferential segment disposed within the plurality of circumferential segments 150 may be non-coplanar in the deployed configuration.

In some embodiments, the centroid(s) 155 of each strut (e.g., the first struts 152 and the second struts 154) of each circumferential segment disposed within the plurality of circumferential segments 150 may be coplanar in the flat pattern configuration. In some embodiments, the centroid(s) 155 of each strut (e.g., the first struts 152, the second struts 154) of each circumferential segment of the plurality of circumferential segments 150 may lie in a plane oriented at an oblique angle 108 relative to the central longitudinal axis 106, as viewed in the flat pattern configuration. In some embodiments, the peaks 156 of each circumferential segment of the plurality of circumferential segments 150 may touch and/or lie in a plane oriented at an oblique angle relative to the central longitudinal axis 106, as viewed in the flat pattern configuration. In some embodiments, the valleys 158 of each circumferential segment of the plurality of circumferential segments 150 may touch and/or lie in a plane oriented at an oblique angle relative to the central longitudinal axis 106, as viewed in the flat pattern configuration.

In some embodiments, the plurality of circumferential segments 150 may be oriented at the oblique angle 108 relative to the central longitudinal axis 106, as viewed in the flat pattern configuration. In some embodiments, the oblique angle 108 may be between about 45 degrees and about 85 degrees. In some embodiments, the oblique angle 108 may be between about 60 degrees and about 85 degrees. In some embodiments, the oblique angle 108 may be between about 70 degrees and about 85 degrees. In one non-limiting example, the oblique angle 108 may be about 79 degrees. Other configurations are also contemplated.

In some embodiments, the peaks 156 and the valleys 158 of adjacent circumferential segments of the plurality of circumferential segments 150 may extend and/or may be aligned helically along the length of the endoprosthesis 100 and/or the body portion 120. In some embodiments, the peaks 156 and the valleys 158 of adjacent circumferential segments of the plurality of circumferential segments 150 may extend and/or may be aligned helically along and/or around the central longitudinal axis 106. In some embodiments, the peaks 156 and the valleys 158 of each circumferential segment of the plurality of circumferential segments 150 may extend and/or may be aligned helically along the length of the endoprosthesis 100 and/or the body portion 120. In some embodiments, the peaks 156 and the valleys 158 of each circumferential segment of the plurality of circumferential segments 150 may extend and/or may be aligned helically along and/or around the central longitudinal axis 106. Other configurations are also contemplated.

In some embodiments, the peaks 156 and the valleys 158 of adjacent circumferential segments of the plurality of circumferential segments 150 may be aligned with each other at an oblique angle relative to the central longitudinal axis 106 in the flat pattern configuration along the length of the endoprosthesis 100 and/or the body portion 120. In some embodiments, the peaks 156 and the valleys 158 of each circumferential segment of the plurality of circumferential segments 150 may be aligned with each other at an oblique angle relative to the central longitudinal axis 106 in the flat pattern configuration along the length of the endoprosthesis 100 and/or the body portion 120. For example, the peaks 156 of one circumferential segment of the plurality of circumferential segments 150 may be aligned at an oblique angle relative to the central longitudinal axis 106 in the flat pattern configuration with the peaks 156 of an immediately adjacent circumferential segment of the plurality of circumferential segments 150. Similarly, the valleys 158 of one circumferential segment of the plurality of circumferential segments 150 may be aligned at an oblique angle relative to the central longitudinal axis 106 in the flat pattern configuration with the valleys 158 of an immediately adjacent circumferential segment of the plurality of circumferential segments 150. Other configurations are also contemplated.

In some embodiments, the plurality of circumferential segments 150 may be oriented substantially parallel to each other in the flat pattern configuration. For example, in the flat pattern configuration, planes containing centroids 155 of each circumferential segment of the plurality of circumferential segments 150 may be oriented parallel to each other. In another example, in the flat pattern configuration, planes containing and/or touching the peaks 156 of each circumferential segment of the plurality of circumferential segments 150 may be oriented parallel to each other. In another example, in the flat pattern configuration, planes containing and/or touching the valleys 158 of each circumferential segment of the plurality of circumferential segments 150 may be oriented parallel to each other.

In some embodiments, each adjacent pair of circumferential segments of the plurality of circumferential segments 150 defines an axial spacing therebetween. The axial spacing may be measured perpendicular to a plane or planes containing the centroids of one or both circumferential segments of each adjacent pair of circumferential segments of the plurality of circumferential segments 150 in the flat pattern configuration. In some embodiments, the axial spacing may be defined between the valleys 158 of one circumferential segment and the peaks 156 of an immediately adjacent circumferential segment in the flat pattern configuration. For the purpose of this disclosure, the axial spacing shall be understood to not include any axial overlap with the first struts 152 and/or the second struts 154 of any given circumferential segment in the flat pattern configuration.

In some embodiments, the axial spacing between adjacent pairs of circumferential segments of the plurality of circumferential segments 150 may be uniform along the length of the endoprosthesis 100 and/or the body portion 120 in the flat pattern configuration. In some embodiments, adjacent pairs of circumferential segments of the plurality of circumferential segments 150 are equally and/or uniformly spaced apart along the length of the endoprosthesis 100 and/or the body portion 120 in the flat pattern configuration. In some embodiments, the axial spacing between adjacent pairs of circumferential segments of the plurality of circumferential segments 150 may vary along the length of the endoprosthesis 100 and/or the body portion 120. In some embodiments, adjacent pairs of circumferential segments of the plurality of circumferential segments 150 are unequally and/or non-uniformly spaced apart along the length of the endoprosthesis 100 and/or the body portion 120. In some embodiments, the axial spacing between adjacent pairs of circumferential segments of the plurality of circumferential segments 150 may increase along the length of the endoprosthesis 100 and/or the body portion 120. In some embodiments, the axial spacing between adjacent pairs of circumferential segments of the plurality of circumferential segments 150 may decrease along the length of the endoprosthesis 100 and/or the body portion 120. Other configurations are also contemplated.

In some embodiments, the peaks 156 and the valleys 158 of each circumferential segment of the plurality of circumferential segments 150 may define a circumferential segment height for that circumferential segment measured perpendicular to a first plane containing and/or touching the peaks 156 and a second plane containing and/or touching the valleys 158 within a given circumferential segment of the plurality of circumferential segments 150 in the flat pattern configuration. In some embodiments, the circumferential segment height may be uniform along the length of the endoprosthesis 100 and/or the body portion 120, and/or among the plurality of circumferential segments 150. In some embodiments, the circumferential segment height may vary along the length of the endoprosthesis 100 and/or the body portion 120, and/or among the plurality of circumferential segments 150. In some embodiments, the circumferential segment height may increase along the length of the endoprosthesis 100 and/or the body portion 120, and/or among the plurality of circumferential segments 150 from the first body end 121 toward and/or to the second body end 129. In some embodiments, the circumferential segment height may decrease along the length of the endoprosthesis 100 and/or the body portion 120, and/or among the plurality of circumferential segments 150 from the first body end 121 toward and/or to the second body end 129.

In some embodiments, the first circumferential end segment 130 may comprise a greater quantity of peaks 136 and valleys 138 than the second circumferential end segment 140 has peaks 146 and valleys 148 and/or the plurality of circumferential segments 150 has peaks 156 and valleys 158. Such a configuration may increase outward radial force within the first circumferential end segment 130 compared to the second circumferential end segment 140 and/or the plurality of circumferential segments 150.

In some embodiments, the second circumferential end segment 140 may comprise a greater quantity of peaks 146 and valleys 148 than the first circumferential end segment 130 has peaks 136 and valleys 138 and/or the plurality of circumferential segments 150 has peaks 156 and valleys 158. Such a configuration may increase outward radial force within the second circumferential end segment 140 compared to the first circumferential end segment 130 and/or the plurality of circumferential segments 150.

In some embodiments, the plurality of circumferential segments 150 may comprise a greater quantity of peaks 156 and valleys 158 than the first circumferential end segment 130 has peaks 136 and valleys 138 and/or the second circumferential end segment 140 has peaks 146 and valleys 148. Such a configuration may increase outward radial force within the plurality of circumferential segments 150 compared to the first circumferential end segment 130 and/or the second circumferential end segment 140. In some embodiments, selected circumferential segments within the plurality of circumferential segments 150 may comprise a greater quantity of peaks 156 and valleys 158 than other circumferential segments within the plurality of circumferential segments 150, thereby varying outward radial force along and/or within the plurality of circumferential segments 150. For example, the endoprosthesis 100 may be configured such that selected and/or specific locations may have increased outward radial force. Other configurations are also contemplated.

In some embodiments, the endoprosthesis 100 may comprise a flared portion 160, selected aspects of which are illustrated in FIG. 3, extending between the body portion 120 and the first end 102, as seen in FIG. 4. The flared portion 160 may have a knitted structure, fabricated from the single wire 110 interwoven with itself. In at least some embodiments, the endoprosthesis 100 may be constructed by forming the body portion 120 first and then continuing on to the flared portion 160. In some alternative embodiments, the endoprosthesis 100 may be constructed by forming the flared portion 160 first and then continuing on to the body portion 120.

In some embodiments, the flared portion 160 may comprise and/or the single wire 110 may form a plurality of knitted circumferential segments 170 (e.g., circumferential rows) extending circumferentially around the central longitudinal axis 106 from the body portion 120 and/or the first body end 121 to the first end 102.

The flared portion 160 may comprise a plurality of knitted circumferential segments 170a, 170b, 170c, 170d, etc. (collectively, the plurality of knitted circumferential segments 170) extending circumferentially around the central longitudinal axis 106. The flared portion 160 may comprise any number of knitted circumferential segments 170 as desired. For example, the number of knitted circumferential segments 170 may be selected to achieve a desired length of the flared portion 160 and/or the endoprosthesis 100. In some embodiments, the flared portion 160 and/or the plurality of knitted circumferential segments 170 may comprise a first knitted circumferential segment 170a disposed immediately adjacent the first circumferential end segment 130 of the body portion 120 (e.g., FIGS. 4, 4A, and 6-12, ).

In some embodiments, the first knitted circumferential segment 170a may comprise a first plurality of loops 172 comprising loops 172a, 172b, 172c, etc. (collectively, ref. 172). The first plurality of loops 172 may each include a loop portion 174a, 174b, 174c, etc. (collectively, ref. 174) and an overlapping base portion 176a, 176b, 176c, etc. (collectively, ref. 176), respectively. The overlapping base portions 176a, 176b, 176c, etc. may be understood as the portion of the first plurality of loops 172 in which one segment of the single wire 110 overlaps or crosses over a second segment of the single wire 110, with the segment of the single wire 110 forming the loop portions 174a, 174b, 174c, etc. extending therebetween. Adjacent loops (e.g., loops 172a and 172b; loops 172b and 172c; etc.) of the first plurality of loops 172 may be interconnected by rung sections 178a, 178b, etc. (collectively, ref. 178). For example, the rung section 178a may extend between the overlapping base portion 176a of a first loop 172a and the overlapping base portion 176b of a second loop 172b.

A second knitted circumferential segment 170b may comprise a second plurality of loops 180 comprising loops 180a, 180b, 180c, etc. (collectively, ref. 180). The second plurality of loops 180 may each include a loop portion 182a, 182b, 182c, etc. (collectively, ref. 182) and an overlapping base portion 184a, 184b, 184c, etc. (collectively, ref. 184), respectively. The overlapping base portions 184a, 184b, 184c, etc. may be understood as the portion of the second plurality of loops 180 in which one segment of the single wire 110 overlaps or crosses over a second segment of the single wire 110, with the segment of the single wire 110 forming the loop portions 182a, 182b, 182c, etc. extending therebetween. Adjacent loops (e.g., loops 180a and 180b; loops 180b and 180c; etc.) of the second plurality of loops 180 may be interconnected by rung sections 186a, 186b, etc. (collectively, ref. 186). For example, the rung section 186a may extend between the overlapping base portion 184a of a first loop 180a and the overlapping base portion 184b of a second loop 180b.

As the flared portion 160 is constructed (e.g., knitted), the loop portion 174a of the first knitted circumferential segment 170a may be wrapped around the overlapping base portion 184a of the second knitted circumferential segment 170b, and/or the first loop 180a of the second knitted circumferential segment 170b may be interwoven through the loop portion 174a of the first loop 172a of the first knitted circumferential segment 170a. Similarly, the loop portion 174b of the first knitted circumferential segment 170a may be wrapped around the overlapping base portion 184b of the second knitted circumferential segment 170b, and/or the second loop 180b of the second knitted circumferential segment 170b may be interwoven through the loop portion 174b of the second loop 172b of the first knitted circumferential segment 170a. This construction method and/or arrangement may be repeated as desired and/or necessary to form the plurality of knitted circumferential segments 170.

FIGS. 4, 5, and 6, schematically illustrate selected aspects of example configurations of the endoprosthesis 100. It shall be understood that while various aspects may be shown in different example configurations disclosed herein, configurations are contemplated within the scope of the disclosure that combine various aspects from different example configurations unless explicitly stated to the contrary.

The endoprosthesis 100 may be configured to shift between a delivery configuration and a deployed configuration. In some embodiments, the endoprosthesis 100 may be configured to be disposed within a delivery system and/or a delivery sheath in the delivery configuration. In some embodiments, the endoprosthesis 100 may be configured to be disposed between an inner tubular member and an outer tubular member of the delivery system and/or the delivery sheath in the delivery configuration. In some embodiments, the inner tubular member and the outer tubular member may be configured to translate relative to each other to deploy the endoprosthesis 100. Other configurations are also contemplated.

The endoprosthesis 100 may be configured to shift toward and/or to the deployed configuration when unconstrained. In some embodiments, the endoprosthesis 100 may be configured to self-expand from the delivery configuration to the deployed configuration. In some embodiments, the endoprosthesis 100 may be self-biased toward the deployed configuration. In some embodiments, the endoprosthesis 100 may be balloon expandable from the delivery configuration to the deployed configuration. Other configurations are also contemplated. In at least some embodiments, the endoprosthesis 100 may be radially expanded in the deployed configuration compared to the delivery configuration.

In some embodiments, the endoprosthesis 100 may extend axially along the central longitudinal axis 106 between a first end 102 and a second end 104. In some embodiments, the endoprosthesis 100 may comprise a body portion 120 as described herein. In some embodiments, the endoprosthesis 100 may comprise a flared portion 160 extending between the body portion 120 and the first end 102, as seen in FIGS. 4, 5, and 6, In some embodiments, the body portion 120 and the flared portion 160 may be formed from the single wire 110 extending from the first end 102 to the second end 104. As discussed herein, the single wire 110 may form the first circumferential end segment 130, the second circumferential end segment 140, and the plurality of circumferential segments 150 of the body portion 120, as well as the plurality of knitted circumferential segments 170 of the flared portion 160.

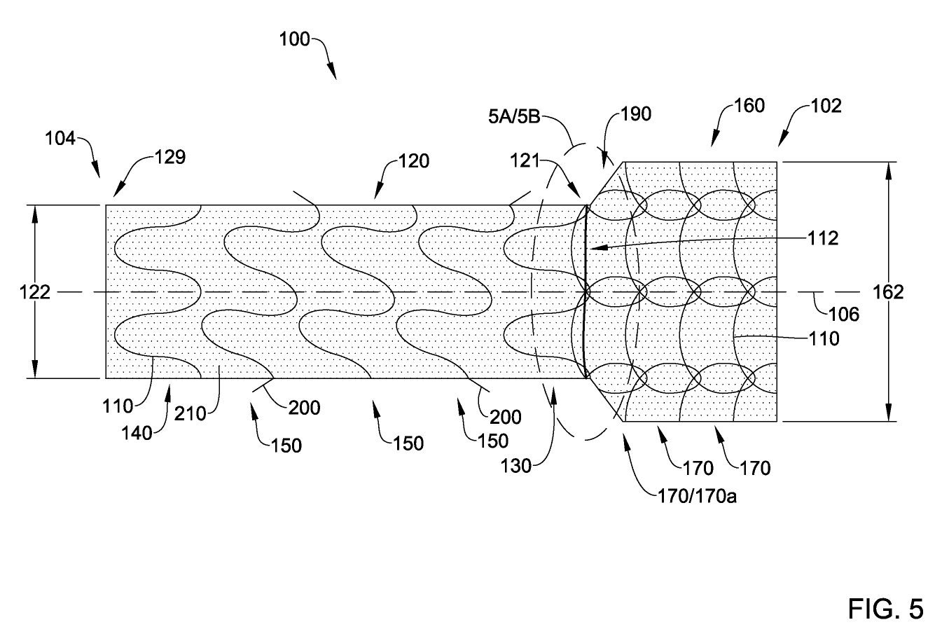

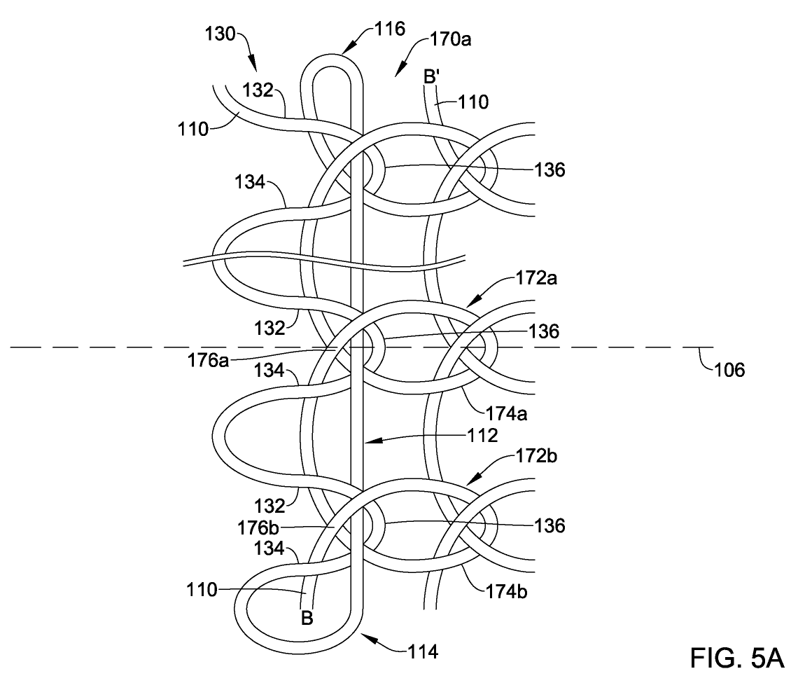

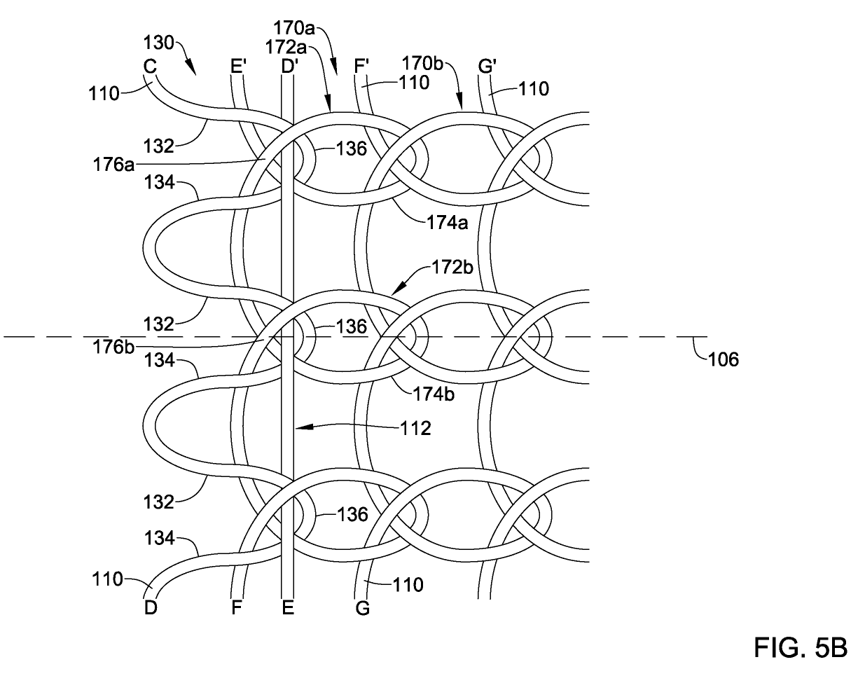

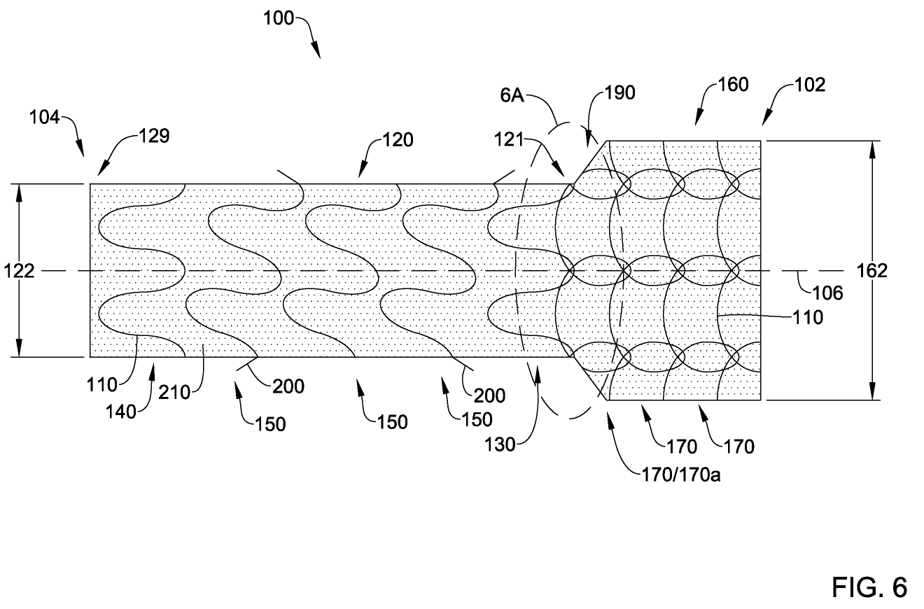

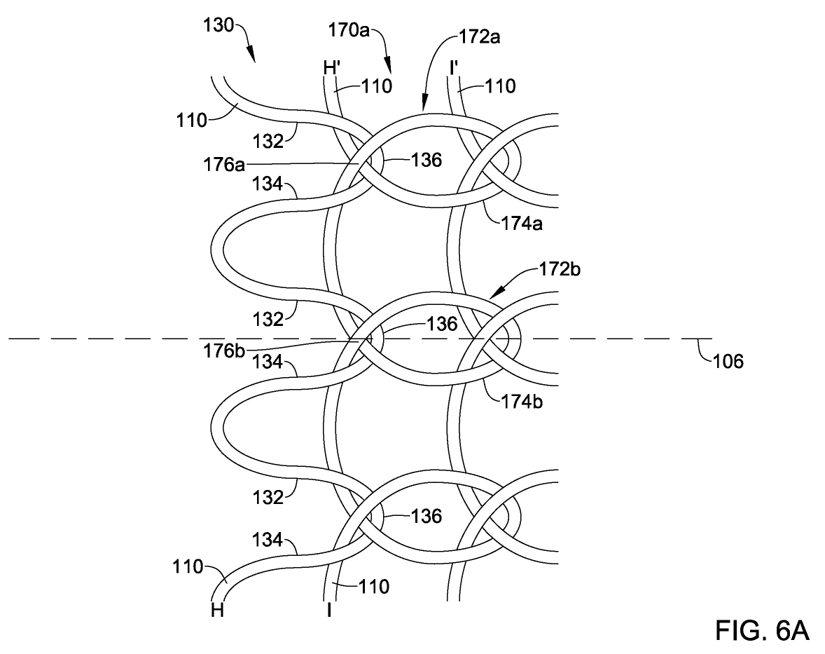

In some embodiments, the single wire 110 may comprise a transition region 112 extending from the body portion 120 to the flared portion 160. In some embodiments, the transition region 112 of the single wire 110 may extend from the first circumferential end segment 130 of the body portion 120 to the first knitted circumferential segment 170a of the flared portion 160. Various configurations and/or details related to the single wire 110, the transition region 112, and/or the transition between the body portion 120 and the flared portion 160 are illustrated in the detail views of FIG. 4A associated with FIG. 4, FIGS. 5A and 5B associated with FIG. 5, and FIG. 6A associated with FIG. 6. It should be noted that some elements, such as the polymeric covering 210 (discussed in more detail below), are not shown in FIGS. 4A, 5A, 5B, and 6A, in the interest of clarity.

In some embodiments, the transition region 112 of the single wire 110 may extend in a generally axial direction from the first circumferential end segment 130 to the first knitted circumferential segment 170a without overlapping, crossing over, and/or intersecting any portion of the single wire 110 disposed within the first circumferential end segment 130, such that the first knitted circumferential segment 170a of the plurality of knitted circumferential segments 170 and/or of the flared portion 160 may be axially spaced apart from the first circumferential end segment 130, as seen in FIG. 4A. For reference, FIG. 4A schematically illustrates the first circumferential end segment 130, wherein a visible (e.g., front-facing) portion of the first circumferential end segment 130 is shown in solid lines and a hidden portion of the first circumferential end segment 130 that extends around the central longitudinal axis 106 out of view is shown in broken lines. Additionally, only a visible (e.g., front-facing) portion of the first knitted circumferential segment 170a is shown, but it shall be understood that a portion of the first knitted circumferential segment 170a also extends around the central longitudinal axis 106 out of view (e.g., the first knitted circumferential segment 170a is shown incomplete). Accordingly, the single wire 110 shall be understood to follow a continuous path from A to A’, etc. even though said continuous path is not visible. In some embodiments, the transition region 112 of the single wire 110 may extend generally parallel with the central longitudinal axis 106.

In some embodiments, the transition region 112 of the single wire 110 may extend circumferentially and/or may be interwoven through the first circumferential end segment 130, as seen in FIGS. 5A and 5B. In some embodiments, the transition region 112 of the single wire 110 may extend circumferentially through and/or may be interwoven through the peaks 136 of the first circumferential end segment 130 and/or between the first struts 132 and the second struts 134 of the first circumferential end segment 130. In some embodiments, the first knitted circumferential segment 170a of the plurality of knitted circumferential segments 170 and/or of the flared portion 160 may be interwoven with the transition region 112 of the single wire 110 and/or the first circumferential end segment 130. For example, the transition region 112 of the single wire 110 may pass through the loop portion 174a of the first loop 172a proximate the overlapping base portion 176a, through the loop portion 174b of the second loop 172b proximate the overlapping base portion 176b, etc.

In some embodiments, the transition region 112 of the single wire 110 may change and/or reverse circumferential direction at least once from the first circumferential end segment 130 to the first knitted circumferential segment 170a of the plurality of knitted circumferential segments 170 and/or of the flared portion 160, as seen in FIG. 5A. It shall be understood that FIG. 5A is highly schematic and is not intended to illustrate a complete construction or a full circumference of the endoprosthesis 100. For example, only a portion of the first knitted circumferential segment 170a is shown. It shall be understood that a portion of the first knitted circumferential segment 170a also extends around the central longitudinal axis 106 out of view (e.g., the first knitted circumferential segment 170a is shown incomplete). Accordingly, the single wire 110 shall be understood to follow a continuous path from B to B’, even though said continuous path is not visible. In some embodiments, the single wire 110 may change circumferential direction from the first circumferential end segment 130 to the transition region 112, as seen at ref. 114. In some embodiments, the single wire 110 may change circumferential direction from the transition region 112 to the first knitted circumferential segment 170a, as seen at ref. 116. Other configurations are also contemplated.

In some embodiments, the transition region 112 of the single wire 110 may extend in one continuous circumferential direction from the first circumferential end segment 130 to the first knitted circumferential segment 170a of the plurality of knitted circumferential segments 170 and/or of the flared portion 160, as seen in FIG. 5B. Similar to FIG. 5A, it shall be understood that FIG. 5B is highly schematic and illustrates an incomplete portion of the endoprosthesis 100. It shall be understood that a portion of the first circumferential end segment 130, a portion of the transition region 112 of the single wire 110, a portion of the first knitted circumferential segment 170a, etc. each extend around the central longitudinal axis 106 out of view (e.g., the elements are shown incomplete). Accordingly, the single wire 110 shall be understood to follow a continuous path from D to D’, from E to E’, from F to F’, and from G to G’, etc. even though said continuous path is not visible.

In some embodiments, the transition region 112 of the single wire 110 may extend from the first circumferential end segment 130 to the first knitted circumferential segment 170a of the plurality of knitted circumferential segments 170 and/or of the flared portion 160 such that the first knitted circumferential segment 170a of the plurality of knitted circumferential segments 170 and/or of the flared portion 160 may be interwoven with the first circumferential end segment 130, as seen in FIG. 6A. It is noted while that the transition region 112 of the single wire 110 is not expressly shown in FIG. 6A, the transition region 112 may be understood to be present out of view. Similar to FIG. 5B above, the transition region 112 of the single wire 110 may extend in one continuous circumferential direction from the first circumferential end segment 130 to the first knitted circumferential segment 170a of the plurality of knitted circumferential segments 170 and/or of the flared portion 160. Additionally, it shall be understood that a portion of the first circumferential end segment 130 and a portion of the first knitted circumferential segment 170a, etc. each extend around the central longitudinal axis 106 out of view (e.g., the elements are shown incomplete). Accordingly, the single wire 110 shall be understood to follow a continuous path from H to H’, from I to I’, etc. even though said continuous path is not visible. In some embodiments, the transition region 112 may be disposed between H and H’. In some alternative embodiments, the transition region 112 may be configured to change circumferential direction from the first circumferential end segment 130 to the first knitted circumferential segment 170a of the plurality of knitted circumferential segments 170 and/or of the flared portion 160 such that the single wire 110 follows a continuous path from H to I (or other similar paths) instead of from H to H’.

Returning to FIGS. 4, 5, and 6, in some embodiments, the endoprosthesis 100 may comprise a transition portion 190 extending from the body portion 120 to the flared portion 160. The transition portion 190 may be formed from the single wire 110. The transition portion 190 may angle radially outward from the body portion 120 toward and/or to the flared portion 160 in the deployed configuration of the endoprosthesis 100. In some embodiments, the transition portion 190 may comprise the transition region 112 of the single wire 110. In some embodiments, the transition portion 190 may comprise at least one knitted circumferential segment of the plurality of knitted circumferential segments 170. In some embodiments, the transition portion 190 may comprise two or more knitted circumferential segments of the plurality of knitted circumferential segments 170. Other configurations are also contemplated.

In some embodiments, the body portion 120 may have a first outer diameter 122. In some embodiments, the first outer diameter 122 of the body portion 120 may be generally uniform and/or generally constant along the length of the body portion 120. In some embodiments, the first outer diameter 122 of the body portion 120 may be between about 2 millimeters and about 30 millimeters, between about 4 millimeters and about 25 millimeters, between about 6 millimeters and about 20 millimeters, between about 8 millimeters and about 15 millimeters, etc. In one non-limiting example, the first outer diameter 122 of the body portion 120 may be about 10 millimeters. Other configurations are also contemplated.

In some embodiments, the flared portion 160 may have a second outer diameter 162. In some embodiments, the second outer diameter 162 of the flared portion 160 may be generally uniform and/or generally constant along the length of the flared portion 160. The second outer diameter 162 may be greater than the first outer diameter 122. In some embodiments, the second outer diameter 162 may be about 20% greater than the first outer diameter 122. In some embodiments, the second outer diameter 162 may be about 30% greater than the first outer diameter 122. In some embodiments, the second outer diameter 162 may be about 40% greater than the first outer diameter 122. In some embodiments, the second outer diameter 162 may be about 50% greater than the first outer diameter 122. Other configurations are also contemplated. In some embodiments, the second outer diameter 162 of the flared portion 160 may be between about 2.2 millimeters and about 45 millimeters, between about 4.8 millimeters and about 37.5 millimeters, between about 7.2 millimeters and about 30 millimeters, between about 9.6 millimeters and about 22.5 millimeters, etc. In one non-limiting example, the second outer diameter 162 of the flared portion 160 may be about 21 millimeters. Other configurations are also contemplated.

In some embodiments, the overall length of the endoprosthesis 100 may be about 100 millimeters to about 150 millimeters. In some embodiments, the overall length of the endoprosthesis 100 may be about 110 millimeters to about 140 millimeters. In some embodiments, the overall length of the endoprosthesis 100 may be about 120 millimeters to about 130 millimeters. Other configurations are also contemplated.

In some embodiments, the endoprosthesis 100 may be configured to minimize and/or avoid foreshortening and/or a change in the overall length of the endoprosthesis 100 as or the endoprosthesis 100 shifts between the delivery configuration and the deployed configuration. In some embodiments, the overall length of the endoprosthesis 100 from the first end 102 to the second end 104 may be configured to change by less than 10% when shifting between the delivery configuration and the deployed configuration. In some embodiments, the overall length of the endoprosthesis 100 from the first end 102 to the second end 104 may be configured to change by less than 7.5% when shifting between the delivery configuration and the deployed configuration. In some embodiments, the overall length of the endoprosthesis 100 from the first end 102 to the second end 104 may be configured to change by less than 5% when shifting between the delivery configuration and the deployed configuration. In some embodiments, the overall length of the endoprosthesis 100 from the first end 102 to the second end 104 may be configured to change by less than 2.5% when shifting between the delivery configuration and the deployed configuration. In some embodiments, the overall length of the endoprosthesis 100 from the first end 102 to the second end 104 may be configured to change by less than 1% when shifting between the delivery configuration and the deployed configuration. Other configurations are also contemplated.

In some embodiments, the body portion 120 may have a body length of about 80 millimeters to about 120 millimeters. In some embodiments, the body length may be about 90 millimeters to about 110 millimeters. In one non-limiting example, the body length may be about 100 millimeters. Other configurations are also contemplated.

In some embodiments, the flared portion 160 may have a flare length of about 12 millimeters to about 40 millimeters. In some embodiments, the flare length may be about 15 millimeters to about 35 millimeters. In some embodiments, the flare length may be about 18 millimeters to about 32 millimeters. In some embodiments, the flare length may be about 22 millimeters to about 28 millimeters. In some embodiments, the flare length may include the transition portion 190. In one non-limiting example, the flare length, including the transition portion 190, may be about 25 millimeters. Other configurations are also contemplated.

In some embodiments, the endoprosthesis 100 and/or the body portion 120 may comprise one or more anti-migration elements disposed along the body length of the endoprosthesis 100 and/or the body portion 120. In some embodiments, at least one circumferential segment of the plurality of circumferential segments 150 may comprise an anti-migration element 200 projecting radially outward from the at least one circumferential segment of the plurality of circumferential segments 150 and/or an outer surface of the endoprosthesis 100 and/or the body portion 120. In some embodiments, at least one circumferential segment of the plurality of circumferential segments 150 may comprise a plurality of anti-migration elements projecting radially outward from the at least one circumferential segment of the plurality of circumferential segments 150 and/or the outer surface of the endoprosthesis 100 and/or the body portion 120. The one or more anti-migration elements and/or the anti-migration element 200 of the at least one circumferential segment of the plurality of circumferential segments 150 may be formed from the single wire 110. For example, each anti-migration element may be formed from a peak 156 (e.g., FIG. 2) or a valley 158 (e.g., FIG. 2) that may be deflected, bent, and/or formed to extend radially outward from the at least one circumferential segment of the plurality of circumferential segments 150 and/or the outer surface of the endoprosthesis 100 and/or the body portion 120.

In some embodiments, at least one anti-migration element may project radially outward and toward the second end 104 (e.g., at a non-zero angle relative to the outer surface of the endoprosthesis 100 and/or the body portion 120) and/or at least one anti-migration element may project radially outward and toward the first end 102 (e.g., at a non-zero angle relative to the outer surface of the endoprosthesis 100 and/or the body portion 120). Other configurations are also contemplated.

The endoprosthesis 100 may comprise a polymeric covering 210, shown in the figures via dotted shading, coupled to the body portion 120 and the flared portion 160 and the transition portion 190, and/or the single wire 110. In some embodiments, the polymeric covering 210 may be fixedly attached to the body portion 120 and the flared portion 160 and the transition portion 190, and/or the single wire 110, and/or the plurality of circumferential segments 150.

In some embodiments, the polymeric covering 210 may extend along an inner surface of the endoprosthesis 100, the single wire 110, and/or the body portion 120, the flared portion 160, and the transition portion 190. In some embodiments, the polymeric covering 210 may extend along an outer surface of the endoprosthesis 100, the single wire 110, and/or the body portion 120, the flared portion 160, and the transition portion 190. In at least some embodiments, the single wire 110, and/or the body portion 120, the flared portion 160, and the transition portion 190 may be embedded within the polymeric covering 210. In some embodiments, the anti-migration element 200, the plurality of anti-migration elements, etc. may be covered by and/or embedded within the polymeric covering 210. Other configurations, including combinations thereof, are also contemplated. Some suitable but non-limiting examples of polymeric materials for the polymeric covering 210 are discussed below.

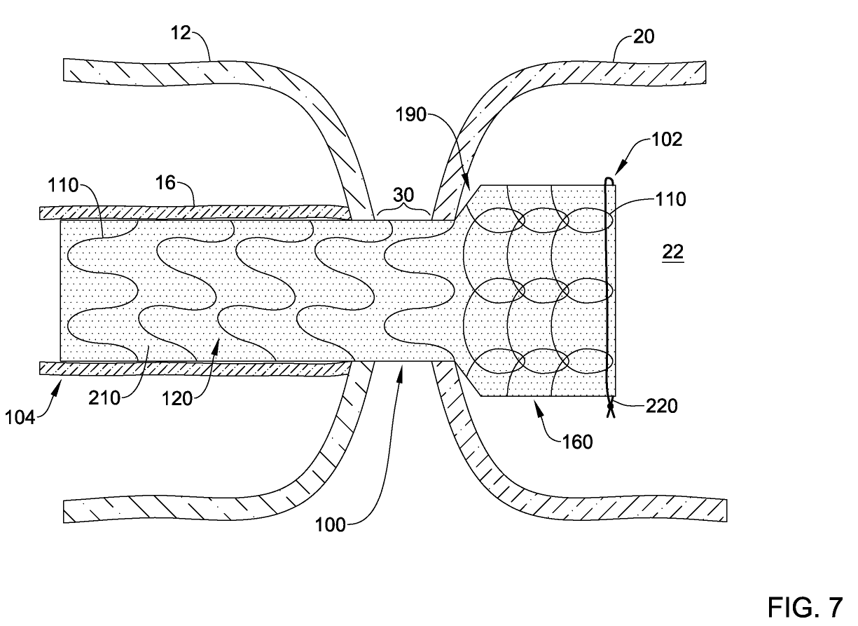

In some embodiments, the body portion 120 of the endoprosthesis 100 may be adapted, sized, and/or configured to be disposed within a first body lumen and the flared portion 160 of the endoprosthesis 100 may be adapted, sized, and/or configured to be disposed within a second body lumen, as seen in FIG. 7. In at least some embodiments, the first body lumen may be a duct of the biliary system 10 (e.g., the hepatic ducts 16) of a patient. In at least some embodiments, the second body lumen may be the stomach 20 of the patient. In some embodiments, the body portion 120 of the endoprosthesis 100 may be adapted, sized, and/or configured to extend through a wall of the second body lumen (e.g., the stomach 20) and/or through a wall of the first body lumen (e.g., the duct of the biliary system 10, the hepatic ducts 16, etc.).

In some embodiments, the flared portion 160 of the endoprosthesis 100 may be adapted, sized, and/or configured to extend and/or to project into an interior space 22 formed within the second body lumen (e.g., the stomach 20) such that the first end 102 of the endoprosthesis 100 is spaced apart from the wall of the second body lumen (e.g., the stomach 20). In some embodiments, the transition portion 190 of the endoprosthesis 100 may be adapted, sized, and/or configured to be engaged with and/or positioned against the wall of the second body lumen (e.g., the stomach 20). In some embodiments, the transition portion 190 and/or the flared portion 160 may be adapted, sized, and/or configured to prevent migration of the endoprosthesis 100 toward the first body lumen (e.g., the duct of the biliary system 10, the hepatic ducts 16, etc.) and/or out of the second body lumen (e.g., the stomach 20).

In some embodiments, the body portion 120 of the endoprosthesis 100 may be adapted, sized, and/or configured to span a gap 30 (e.g., the peritoneal space) between the first body lumen (e.g., the duct of the biliary system 10, the hepatic ducts 16, etc.) and the second body lumen (e.g., the stomach 20). As discussed herein, the endoprosthesis 100 may be fully covered by the polymeric covering 210. The polymeric covering 210 may enable the endoprosthesis 100 to provide a fluid-tight lumen and/or conduit between the first body lumen (e.g., the duct of the biliary system 10, the hepatic ducts 16, etc.) and the second body lumen (e.g., the stomach 20). Additionally, the polymeric covering 210 may prevent leakage within and/or into the gap 30 (e.g., the peritoneal space) between the first body lumen (e.g., the duct of the biliary system 10, the hepatic ducts 16, etc.) and the second body lumen (e.g., the stomach 20), thereby protecting adjacent tissues and/or anatomy.

In some embodiments, the endoprosthesis 100 may comprise a removal suture 220 disposed proximate the first end 102, as seen in FIG. 7. In some embodiments, the removal suture 220 may be interwoven with the first end 102 and/or the flared portion 160. The removal suture 220 may be configured to facilitate and/or aid removal of the endoprosthesis 100 from the patient after deployment. In some embodiments, the removal suture 220 may be at least partially wrapped around the single wire 110. In some embodiments, the removal suture 220 may be configured to be grasped and/or held, such as with a forceps or other grasping tool. In some embodiments, the removal suture 220 may be configured to transfer tension applied thereto to the endoprosthesis 100, the first end 102, and/or the flared portion 160 to facilitate removing the endoprosthesis 100 from the patient after deployment. In some embodiments, the removal suture 220 may be partially embedded within the polymeric covering 210. In some embodiments, the removal suture 220 may be passed through the polymeric covering 210 as it is interwoven with and/or around the single wire 110. In some embodiments, the removal suture 220 may also be interwoven with one or more knitted circumferential segments of the plurality of knitted circumferential segments 170. Other configurations are also contemplated.

In some embodiments, the first circumferential end segment 130 may comprise a double circumferential loop of the single wire 110. In some embodiments, the first circumferential end segment 130, and/or the double circumferential loop thereof, may comprise a first circumferential loop formed from the single wire 110 and a second circumferential loop formed from the single wire 110, wherein the first circumferential loop of the first circumferential end segment 130 and the second circumferential loop of the first circumferential end segment 130 extend circumferentially around the central longitudinal axis 106 in immediate proximity to each other. In some embodiments, the first circumferential loop of the first circumferential end segment 130 may be oriented substantially parallel to the second circumferential loop of the first circumferential end segment 130. In some embodiments, the first circumferential loop and the second circumferential loop of the first circumferential end segment 130 may extend circumferentially around the central longitudinal axis 106 in direct contact with other. In some embodiments, the first circumferential loop of the first circumferential end segment 130 may be fixedly secured directly to the second circumferential loop of the first circumferential end segment 130, such as by welding, soldering, brazing, or adhesive. In some embodiments, the double circumferential loop of the first circumferential end segment 130 may add strength and/or support to the first body end 121 of the endoprosthesis 100 to facilitate removal of the endoprosthesis 100 from the patient after deployment. Other benefits and/or advantages (e.g., preventing radial collapse, improved migration resistance, etc.) are also contemplated.

In some embodiments, the second circumferential end segment 140 may comprise a double circumferential loop of the single wire 110. In some embodiments, the second circumferential end segment 140, and/or the double circumferential loop thereof, may comprise a first circumferential loop formed from the single wire 110 and a second circumferential loop formed from the single wire 110, wherein the first circumferential loop and the second circumferential loop of the second circumferential end segment 140 extend circumferentially around the central longitudinal axis 106 in immediate proximity to each other. In some embodiments, the first circumferential loop of the second circumferential end segment 140 may be oriented substantially parallel to the second circumferential loop of the second circumferential end segment 140. In some embodiments, the first circumferential loop and the second circumferential loop of the second circumferential end segment 140 may extend circumferentially around the central longitudinal axis 106 in direct contact with other. In some embodiments, the first circumferential loop of the second circumferential end segment 140 may be fixedly secured directly to the second circumferential loop of the second circumferential end segment 140, such as by welding, soldering, brazing, or adhesive. In some embodiments, the double circumferential loop of the second circumferential end segment 140 may add strength and/or support to the second end 104 of the endoprosthesis 100 to prevent radial collapse of the second end 104 after deployment. In some embodiments, the polymeric covering 210 may be configured such that the second circumferential end segment 140 is uncovered with the plurality of circumferential segments 150 being covered to allow for potential ingrowth of tissue into and/or around the second circumferential end segment 140 to further improve migration resistance or prevention.

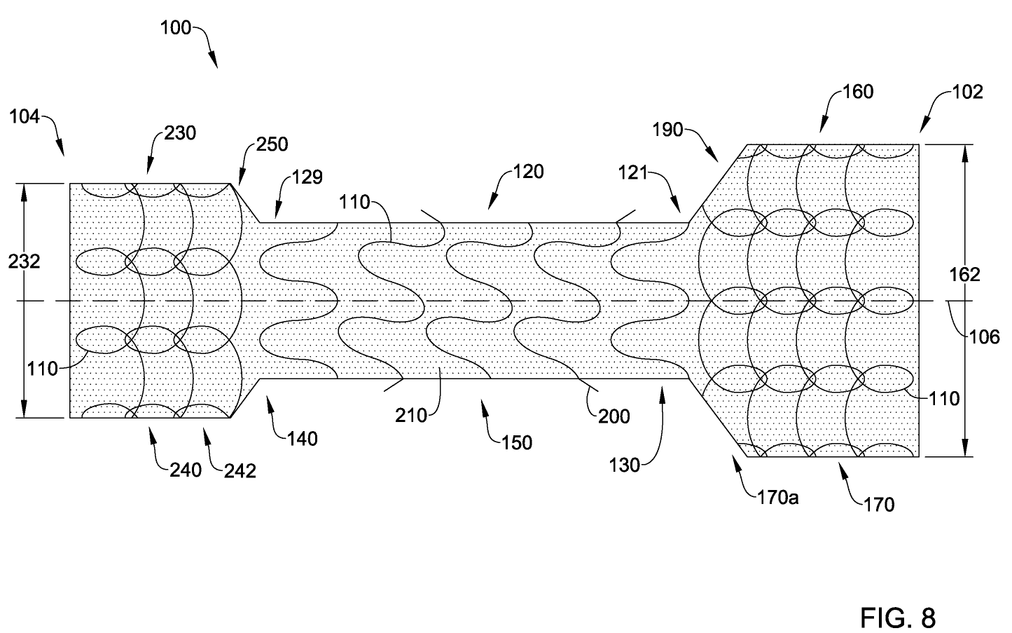

In some embodiments, the endoprosthesis 100 may comprise a second flared portion 230 formed from the single wire 110. The second flared portion 230 may extend between the body portion 120 and the second end 104, as seen in FIG. 8. Accordingly, the body portion 120 may be spaced part from the first end 102 and the second end 104. In at least some embodiments, the second flared portion 230 may have a knitted structure, fabricated from the single wire 110 interwoven with itself.

In some embodiments, the second flared portion 230 may comprise and/or the single wire 110 may form a second plurality of knitted circumferential segments 240 (e.g., circumferential rows) extending circumferentially around the central longitudinal axis 106 from the body portion 120 and/or the second body end 129 to the second end 104. In some embodiments, the second plurality of knitted circumferential segments 240 may be constructed in a similar manner to the plurality of knitted circumferential segments 170. As such, details related to the structure and/or arrangement of the plurality of knitted circumferential segments 170 may also apply to the second plurality of knitted circumferential segments 240 and are not repeated herein.

In some embodiments, the single wire 110 may comprise a second transition region extending from the body portion 120 to the second flared portion 230. In some embodiments, the second transition region may extend from the second circumferential end segment 140 of the body portion 120 to a first knitted circumferential segment 242 of the second plurality of knitted circumferential segments 240 of the second flared portion 230. In some embodiments, details related to the single wire 110, the second transition region, and/or the transition between the body portion 120 and the second flared portion 230 may be similar to the transition region 112 and/or the transition between the body portion 120 and the flared portion 160.

In some embodiments, the second transition region of the single wire 110 may extend in a generally axial direction from the second circumferential end segment 140 to the first knitted circumferential segment 242 of the second plurality of knitted circumferential segments 240 of the second flared portion 230 without overlapping, crossing over, and/or intersecting any portion of the single wire 110 disposed within the second circumferential end segment 140, such that the first knitted circumferential segment 242 of the second plurality of knitted circumferential segments 240 of the second flared portion 230 may be axially spaced apart from the second circumferential end segment 140, similar to the transition region 112 shown in FIG. 4A.

In some embodiments, the second transition region of the single wire 110 may extend circumferentially and/or may be interwoven through the second circumferential end segment 140, similar to the transition region 112 shown in FIGS. 5A-5B. In some embodiments, the second transition region of the single wire 110 may extend circumferentially through and/or may be interwoven through the peaks 146 (e.g., FIG. 2) of the second circumferential end segment 140 and/or between the first struts 142 (e.g., FIG. 2) and the second struts 144 (e.g., FIG. 2) of the second circumferential end segment 140. In some embodiments, the first knitted circumferential segment 242 of the second plurality of knitted circumferential segments 240 of the second flared portion 230 may be interwoven with the second transition region of the single wire 110 and/or the second circumferential end segment 140.

In some embodiments, the second transition region of the single wire 110 may change and/or reverse circumferential direction at least once from the second circumferential end segment 140 to the first knitted circumferential segment 242 of the second plurality of knitted circumferential segments 240 of the second flared portion 230, similar to the transition region 112 shown in FIG. 5A. In some embodiments, the single wire 110 may change circumferential direction from the second circumferential end segment 140 to the second transition region. In some embodiments, the single wire 110 may change circumferential direction from the second transition region to the first knitted circumferential segment 242 of the second plurality of knitted circumferential segments 240 of the second flared portion 230. Other configurations are also contemplated.

In some embodiments, the second transition region of the single wire 110 may extend in one continuous circumferential direction from the second circumferential end segment 140 to the first knitted circumferential segment 242 of the second plurality of knitted circumferential segments 240 of the second flared portion 230, similar to the transition region 112 shown in FIG. 5B.

In some embodiments, the second transition region of the single wire 110 may extend from the second circumferential end segment 140 to the first knitted circumferential segment 242 of the second plurality of knitted circumferential segments 240 of the second flared portion 230 such that the first knitted circumferential segment 242 of the second plurality of knitted circumferential segments 240 of the second flared portion 230 may be interwoven with the second circumferential end segment 140, similar to the transition region 112 described with respect to FIG. 6A. In some alternative embodiments, the second transition region may be configured to change circumferential direction from the second circumferential end segment 140 to the first knitted circumferential segment 242 of the second plurality of knitted circumferential segments 240 of the second flared portion 230.

In some embodiments, the endoprosthesis 100 may comprise a second transition portion 250 extending from the body portion 120 to the second flared portion 230. The second transition portion 250 may be formed from the single wire 110. The second transition portion 250 may angle radially outward from the body portion 120 toward and/or to the second flared portion 230 in the deployed configuration of the endoprosthesis 100. In some embodiments, the second transition portion 250 may comprise the second transition region of the single wire 110. In some embodiments, the second transition portion 250 may comprise at least one knitted circumferential segment of the second plurality of knitted circumferential segments 240. In some embodiments, the second transition portion 250 may comprise two or more knitted circumferential segments of the second plurality of knitted circumferential segments 240. Other configurations are also contemplated.

In some embodiments, the second flared portion 230 may have a third outer diameter 232. In some embodiments, the third outer diameter 232 of the second flared portion 230 may be generally uniform and/or generally constant along the length of the second flared portion 230. In at least some embodiments, the third outer diameter 232 of the second flared portion 230 may be greater than the first outer diameter 122 (e.g., FIG. 4) of the body portion 120 and less than the second outer diameter 162 of the flared portion 160. Other configurations are also contemplated.

In some embodiments, the second flared portion 230 may have a second flare length of about 12 millimeters to about 40 millimeters. In some embodiments, the second flare length may be about 15 millimeters to about 35 millimeters. In some embodiments, the second flare length may be about 18 millimeters to about 32 millimeters. In some embodiments, the second flare length may be about 22 millimeters to about 28 millimeters. In some embodiments, the second flare length may include the second transition portion. In one non-limiting example, the second flare length, including the second transition portion, may be about 25 millimeters. Other configurations are also contemplated.

As discussed herein, at least one circumferential segment of the plurality of circumferential segments 150 may comprise an anti-migration element 200 projecting radially outward from the at least one circumferential segment of the plurality of circumferential segments 150 and/or an outer surface of the endoprosthesis 100 and/or the body portion 120. The one or more anti-migration elements and/or the anti-migration element 200 of the at least one circumferential segment of the plurality of circumferential segments 150 may be formed from the single wire 110.

Similarly, the endoprosthesis 100 may comprise a polymeric covering 210, shown in the figures via dotted shading, coupled to the body portion 120, the flared portion 160, and the second flared portion 230. In some embodiments, the polymeric covering 210 may be fixedly attached to the body portion 120, the flared portion 160, and the second flared portion 230, and/or the single wire 110, and/or the plurality of circumferential segments 150. In some embodiments, the polymeric covering 210 may extend along an inner surface of the endoprosthesis 100, the single wire 110, and/or the body portion 120, the flared portion 160, and the second flared portion 230. In some embodiments, the polymeric covering 210 may extend along an outer surface of the endoprosthesis 100, the single wire 110, and/or the body portion 120, the flared portion 160, and the second flared portion 230. In at least some embodiments, the single wire 110, and/or the body portion 120, the flared portion 160, and the second flared portion 230 may be embedded within the polymeric covering 210. In some embodiments, the anti-migration element 200, the plurality of anti-migration elements, etc. may be covered by and/or embedded within the polymeric covering 210. Other configurations, including combinations thereof, are also contemplated.

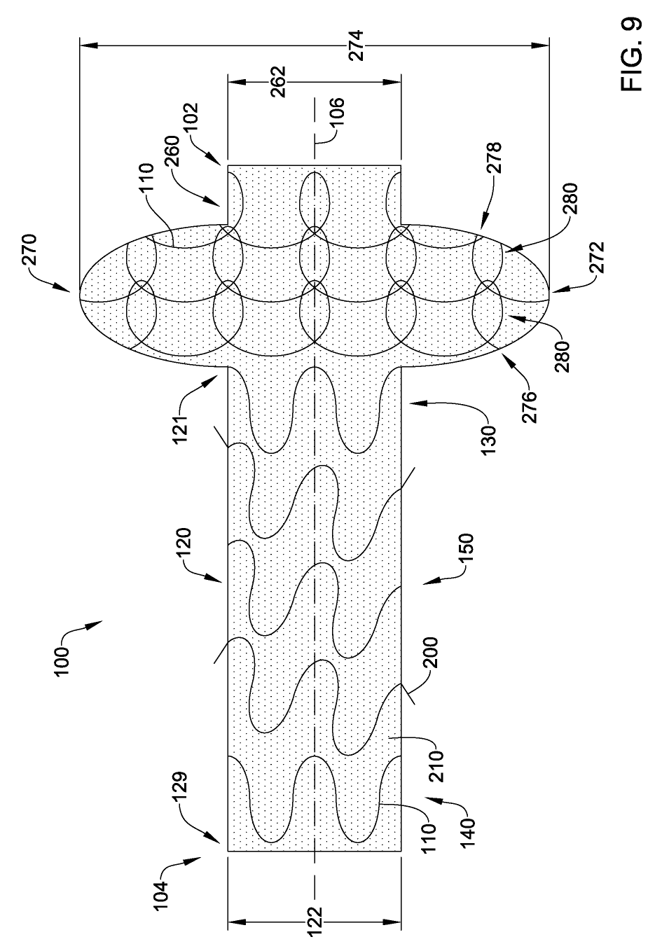

FIGS. 9-12 schematically illustrate selected aspects of example configurations of the endoprosthesis 100 in a side view. It will be appreciated that many, if not all, of the features, characteristics, benefits, advantages, etc. of the endoprosthesis 100 illustrated in FIGS. 2-6A may also apply to the endoprosthesis 100 of FIG. 9. Accordingly, like reference numerals are used to refer to like elements.

Similar to the endoprosthesis 100 described above, the endoprosthesis 100 of FIG. 9 may comprise a body portion 120 extending axially along the central longitudinal axis 106 from the second end 104 toward the first end 102. The endoprosthesis 100 may comprise a first end portion 260 extending axially along the central longitudinal axis 106 from the first end 102 toward the second end 104. The endoprosthesis 100 may comprise a flanged portion 270 extending between the body portion 120 and the first end portion 260. In some embodiments, the body portion 120, the first end portion 260, and the flanged portion 270 may be formed from the single wire 110 extending from the first end 102 to the second end 104. As discussed herein, the single wire 110 may form the first circumferential end segment 130, the second circumferential end segment 140, and the plurality of circumferential segments 150 extending helically around the central longitudinal axis 106 between the first circumferential end segment 130 and the second circumferential end segment 140.

In some embodiments, the flanged portion 270 and/or the first end portion 260 may have a knitted structure, fabricated from the single wire 110 interwoven with itself, similar to the flared portion 160 described herein. In at least some embodiments, the endoprosthesis 100 may be constructed by forming the body portion 120 first and then continuing on to the flanged portion 270 and the first end portion 260. In some alternative embodiments, the endoprosthesis 100 may be constructed by forming the first end portion 260 first and then continuing on to the flanged portion 270 and the body portion 120.

In some embodiments, the flanged portion 270 may comprise and/or the single wire 110 may form a plurality of knitted circumferential segments 280 (e.g., circumferential rows) extending circumferentially around the central longitudinal axis 106 from the body portion 120 and/or the first body end 121 to the first end portion 260. In some embodiments, the plurality of knitted circumferential segments 280 may be constructed in a similar manner to the plurality of knitted circumferential segments 170. As such, details related to the structure and/or arrangement of the plurality of knitted circumferential segments 170 may also apply to the plurality of knitted circumferential segments 280 and are not repeated herein. In at least some embodiments, the plurality of knitted circumferential segments 280 may extend circumferentially around the central longitudinal axis 106 within the first end portion 260 from the flanged portion 270 to the first end 102.

The flanged portion 270 may comprise a medial region 272 having a medial outer diameter 274, wherein the medial outer diameter 274 is greater than the outer diameter (e.g., the first outer diameter 122) of the body portion 120 and the medial outer diameter 274 is greater than an outer diameter 262 of the first end portion 260. In some embodiments, the outer diameter 262 of the first end portion 260 may be similar to the outer diameter of the body portion 120 (e.g., the first outer diameter 122). In some embodiments, the outer diameter 262 of the first end portion 260 may be equal to the outer diameter of the body portion 120 (e.g., the first outer diameter 122). In some embodiments, the outer diameter 262 of the first end portion 260 may be within about 10% of the outer diameter of the body portion 120 (e.g., the first outer diameter 122). In some embodiments, the outer diameter 262 of the first end portion 260 may be within about 5% of the outer diameter of the body portion 120 (e.g., the first outer diameter 122). Other configurations are also contemplated.

In some embodiments, the medial outer diameter 274 may be about 20% greater than the outer diameter of the body portion 120 (e.g., the first outer diameter 122) and/or the outer diameter 262 of the first end portion 260. In some embodiments, the medial outer diameter 274 may be about 30% greater than the outer diameter of the body portion 120 (e.g., the first outer diameter 122) and/or the outer diameter 262 of the first end portion 260. In some embodiments, the medial outer diameter 274 may be about 40% greater than the outer diameter of the body portion 120 (e.g., the first outer diameter 122) and/or the outer diameter 262 of the first end portion 260. In some embodiments, the medial outer diameter 274 may be about 50% greater than the outer diameter of the body portion 120 (e.g., the first outer diameter 122) and/or the outer diameter 262 of the first end portion 260. Other configurations are also contemplated. In some embodiments, the medial outer diameter 274 may be between about 2.2 millimeters and about 45 millimeters, between about 4.8 millimeters and about 37.5 millimeters, between about 7.2 millimeters and about 30 millimeters, between about 9.6 millimeters and about 22.5 millimeters, etc. In one non-limiting example, the medial outer diameter 274 may be about 21 millimeters. Other configurations are also contemplated.

In some embodiments, the outer diameter 262 of the first end portion 260 being smaller than the medial outer diameter 274 may reduce food impaction on the first end 102 of the endoprosthesis 100 while permitting and/or maintaining internal biliary drainage.

The flanged portion 270 may comprise a first transition portion 276 extending from the body portion 120 to the medial region 272. The first transition portion 276 may angle and/or curve radially outward from the body portion 120 toward and/or to the medial region 272 in the deployed configuration of the endoprosthesis 100. The flanged portion 270 may comprise a second transition portion 278 extending from the first end portion 260 to the medial region 272. The second transition portion 278 may angle and/or curve radially outward from the first end portion 260 toward and/or to the medial region 272 in the deployed configuration of the endoprosthesis 100.

The first transition portion 276 and the second transition portion 278 may be formed from the single wire 110. In some embodiments, the first transition portion 276 may comprise at least one knitted circumferential segment of the plurality of knitted circumferential segments 280. In some embodiments, the first transition portion 276 may comprise two or more knitted circumferential segments of the plurality of knitted circumferential segments 280. In some embodiments, the second transition portion 278 may comprise at least one knitted circumferential segment of the plurality of knitted circumferential segments 280. In some embodiments, the second transition portion 278 may comprise two or more knitted circumferential segments of the plurality of knitted circumferential segments 280.

As discussed herein, at least one circumferential segment of the plurality of circumferential segments 150 may comprise an anti-migration element 200 projecting radially outward from the at least one circumferential segment of the plurality of circumferential segments 150 and/or an outer surface of the endoprosthesis 100 and/or the body portion 120. The one or more anti-migration elements and/or the anti-migration element 200 of the at least one circumferential segment of the plurality of circumferential segments 150 may be formed from the single wire 110.

Similarly, the endoprosthesis 100 may comprise a polymeric covering 210, shown in the figures via dotted shading, coupled to the body portion 120, the first end portion 260, and the flanged portion 270. In some embodiments, the polymeric covering 210 may be fixedly attached to the body portion 120, the first end portion 260, and the flanged portion 270, and/or the single wire 110, and/or the plurality of circumferential segments 150 and the plurality of knitted circumferential segments 280. In some embodiments, the polymeric covering 210 may extend along an inner surface of the endoprosthesis 100, the single wire 110, and/or the body portion 120, the first end portion 260, and the flanged portion 270. In some embodiments, the polymeric covering 210 may extend along an outer surface of the endoprosthesis 100, the single wire 110, and/or the body portion 120, the first end portion 260, and the flanged portion 270. In at least some embodiments, the single wire 110, and/or the body portion 120, the first end portion 260, and the flanged portion 270 may be embedded within the polymeric covering 210. In some embodiments, the anti-migration element 200, the plurality of anti-migration elements, etc. may be covered by and/or embedded within the polymeric covering 210. Other configurations, including combinations thereof, are also contemplated.

In some embodiments, the body portion 120 of the endoprosthesis 100 may be adapted, sized, and/or configured to be disposed within a first body lumen, and the first end portion 260 and the flanged portion 270 of the endoprosthesis 100 may be adapted, sized, and/or configured to be disposed within a second body lumen. In at least some embodiments, the first body lumen may be a duct of the biliary system 10 (e.g., the hepatic ducts 16) of a patient. In at least some embodiments, the second body lumen may be the stomach 20 of the patient. In some embodiments, the body portion 120 of the endoprosthesis 100 may be adapted, sized, and/or configured to extend through a wall of the second body lumen (e.g., the stomach 20) and/or through a wall of the first body lumen (e.g., the duct of the biliary system 10, the hepatic ducts 16, etc.).

In some embodiments, the first end portion 260 of the endoprosthesis 100 may be adapted, sized, and/or configured to extend and/or to project into an interior space 22 formed within the second body lumen (e.g., the stomach 20) such that the first end 102 of the endoprosthesis 100 is spaced apart from the wall of the second body lumen (e.g., the stomach 20). In some embodiments, the flanged portion 270 and/or the first transition portion 276 of the endoprosthesis 100 may be adapted, sized, and/or configured to be engaged with and/or positioned against the wall of the second body lumen (e.g., the stomach 20). In some embodiments, the flanged portion 270 and/or the first transition portion 276 may be adapted, sized, and/or configured to prevent migration of the endoprosthesis 100 toward the first body lumen (e.g., the duct of the biliary system 10, the hepatic ducts 16, etc.) and/or out of the second body lumen (e.g., the stomach 20).

In some embodiments, the body portion 120 of the endoprosthesis 100 may be adapted, sized, and/or configured to span the gap 30 (e.g., FIG. 7) between the first body lumen (e.g., the duct of the biliary system 10, the hepatic ducts 16, etc.) and the second body lumen (e.g., the stomach 20). As discussed herein, the endoprosthesis 100 may be fully covered by the polymeric covering 210. The polymeric covering 210 may enable the endoprosthesis 100 to provide a fluid-tight lumen and/or conduit between the first body lumen (e.g., the duct of the biliary system 10, the hepatic ducts 16, etc.) and the second body lumen (e.g., the stomach 20). Additionally, the polymeric covering 210 may prevent leakage within and/or into the gap 30 (e.g., the peritoneal space) between the first body lumen (e.g., the duct of the biliary system 10, the hepatic ducts 16, etc.) and the second body lumen (e.g., the stomach 20), thereby protecting adjacent tissues and/or anatomy.

In some embodiments, the endoprosthesis 100 may comprise a removal suture 220 (e.g., FIG. 7) disposed proximate the first end 102. The removal suture 220 may be configured to facilitate and/or aid removal of the endoprosthesis 100 from the patient after deployment. In some embodiments, the removal suture 220 may be interwoven with the first end 102 and/or the first end portion 260. In some embodiments, the removal suture 220 may be at least partially wrapped around the single wire 110 within the first end portion 260 and/or at the first end 102. In some embodiments, the removal suture 220 may be configured to be grasped and/or held, such as with a forceps or other grasping tool. In some embodiments, the removal suture 220 may be configured to transfer tension applied thereto to the endoprosthesis 100, the first end 102, and/or the first end portion 260 to facilitate removing the endoprosthesis 100 from the patient after deployment. In some embodiments, the removal suture 220 may be partially embedded within the polymeric covering 210. In some embodiments, the removal suture 220 may be passed through the polymeric covering 210 as it is interwoven with the first end 102 and/or the first end portion 260. In some embodiments, the removal suture 220 may also be interwoven with one or more knitted circumferential segments of the plurality of knitted circumferential segments 280. Other configurations are also contemplated.

In some embodiments, the first circumferential end segment 130 may comprise a double circumferential loop of the single wire 110. In some embodiments, the first circumferential end segment 130, and/or the double circumferential loop thereof, may comprise a first circumferential loop formed from the single wire 110 and a second circumferential loop formed from the single wire 110, wherein the first circumferential loop of the first circumferential end segment 130 and the second circumferential loop of the first circumferential end segment 130 extend circumferentially around the central longitudinal axis 106 in immediate proximity to each other. In some embodiments, the first circumferential loop of the first circumferential end segment 130 may be oriented substantially parallel to the second circumferential loop of the first circumferential end segment 130. In some embodiments, the first circumferential loop and the second circumferential loop of the first circumferential end segment 130 may extend circumferentially around the central longitudinal axis 106 in direct contact with other. In some embodiments, the first circumferential loop of the first circumferential end segment 130 may be fixedly secured directly to the second circumferential loop of the first circumferential end segment 130, such as by welding, soldering, brazing, or adhesive. In some embodiments, the double circumferential loop of the first circumferential end segment 130 may add strength and/or support to the first body end 121 of the endoprosthesis 100 to facilitate removal of the endoprosthesis 100 from the patient after deployment. Other benefits and/or advantages (e.g., preventing radial collapse, improved migration resistance, etc.) are also contemplated.

In some embodiments, the second circumferential end segment 140 may comprise a double circumferential loop of the single wire 110. In some embodiments, the second circumferential end segment 140, and/or the double circumferential loop thereof, may comprise a first circumferential loop formed from the single wire 110 and a second circumferential loop formed from the single wire 110, wherein the first circumferential loop and the second circumferential loop of the second circumferential end segment 140 extend circumferentially around the central longitudinal axis 106 in immediate proximity to each other. In some embodiments, the first circumferential loop of the second circumferential end segment 140 may be oriented substantially parallel to the second circumferential loop of the second circumferential end segment 140. In some embodiments, the first circumferential loop and the second circumferential loop of the second circumferential end segment 140 may extend circumferentially around the central longitudinal axis 106 in direct contact with other. In some embodiments, the first circumferential loop of the second circumferential end segment 140 may be fixedly secured directly to the second circumferential loop of the second circumferential end segment 140, such as by welding, soldering, brazing, or adhesive. In some embodiments, the double circumferential loop of the second circumferential end segment 140 may add strength and/or support to the second end 104 of the endoprosthesis 100 to prevent radial collapse of the second end 104 after deployment. In some embodiments, the polymeric covering 210 may be configured such that the second circumferential end segment 140 is uncovered with the plurality of circumferential segments 150 being covered to allow for potential ingrowth of tissue into and/or around the second circumferential end segment 140 to further improve migration resistance or prevention.

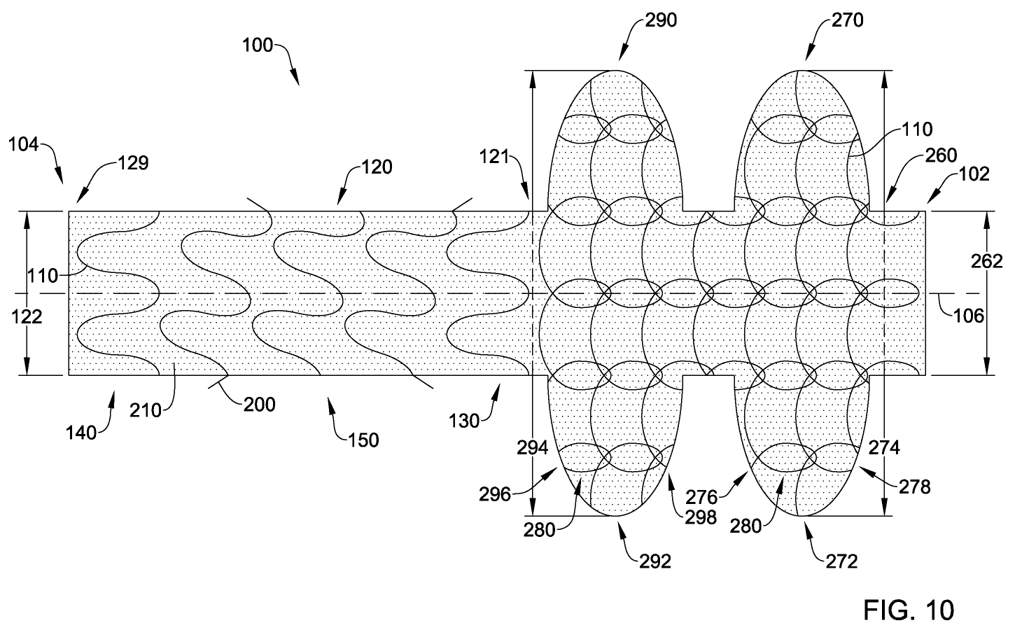

In some embodiments, the endoprosthesis 100 may comprise a paired flange portion 290 disposed proximate the flanged portion 270 and/or between the body portion 120 and the first end portion 260, as seen in FIG. 10. In some embodiments, the flanged portion 270 and the paired flange portion 290 of the endoprosthesis 100 may be considered to form a “double flange” proximate the first end 102 of the endoprosthesis 100. The paired flange portion 290 may be formed from the single wire 110 extending from the first end 102 to the second end 104.

In some embodiments, the paired flange portion 290 may have a knitted structure, fabricated from the single wire 110 interwoven with itself, similar to the flared portion 160 and/or the flanged portion 270 described herein. In at least some embodiments, the endoprosthesis 100 may be constructed by forming the body portion 120 first and then continuing on to the paired flange portion 290, the flanged portion 270, and the first end portion 260. In some alternative embodiments, the endoprosthesis 100 may be constructed by forming the first end portion 260 first and then continuing on to the flanged portion 270, the paired flange portion 290, and the body portion 120.

In some embodiments, the paired flange portion 290 may be formed from the plurality of knitted circumferential segments 280 (e.g., circumferential rows) extending circumferentially around the central longitudinal axis 106 from the body portion 120 and/or the first body end 121 to the first end portion 260. In some embodiments, the plurality of knitted circumferential segments 280 may be constructed in a similar manner to the plurality of knitted circumferential segments 170. As such, details related to the structure and/or arrangement of the plurality of knitted circumferential segments 170 may also apply to the plurality of knitted circumferential segments 280 and are not repeated herein.

The paired flange portion 290 may comprise a medial region 292 having a medial outer diameter 294, wherein the medial outer diameter 294 of the paired flange portion 290 is greater than the outer diameter (e.g., the first outer diameter 122) of the body portion 120 and the medial outer diameter 294 of the paired flange portion 290 is greater than an outer diameter 262 of the first end portion 260. In some embodiments, the medial outer diameter 294 of the paired flange portion 290 may be similar to the medial outer diameter 274 of the flanged portion 270. In some embodiments, the medial outer diameter 294 of the paired flange portion 290 may be equal to the medial outer diameter 274 of the flanged portion 270. Other configurations are also contemplated.

The paired flange portion 290 may comprise a first transition portion 296 and a second transition portion 298 formed similar to the first transition portion 276 and the second transition portion 278 of the flanged portion 270. For example, the first transition portion 296 and the second transition portion 298 may angle and/or curve radially outward toward and/or to the medial region 292 of the paired flange portion 290 in the deployed configuration of the endoprosthesis 100. The first transition portion 296 and the second transition portion 298 of the paired flange portion 290 may be formed from the single wire 110. In some embodiments, the first transition portion 296 of the paired flange portion 290 may comprise at least one knitted circumferential segment of the plurality of knitted circumferential segments 280. In some embodiments, the first transition portion 296 of the paired flange portion 290 may comprise two or more knitted circumferential segments of the plurality of knitted circumferential segments 280. In some embodiments, the second transition portion 298 of the paired flange portion 290 may comprise at least one knitted circumferential segment of the plurality of knitted circumferential segments 280. In some embodiments, the second transition portion 298 of the paired flange portion 290 may comprise two or more knitted circumferential segments of the plurality of knitted circumferential segments 280.

In some embodiments, the paired flange portion 290 may be axially spaced apart from flanged portion 270 by a small amount (considerably less than the length of the body portion 120, for example). In some embodiments, the small amount may be less than 10 millimeters, less than 7.5 millimeters, less than 5 millimeters, less than 2.5 millimeters, etc. In some embodiments, the small amount may be less than an axial length of the flanged portion 270 or the paired flange portion 290. In some embodiments, the small amount may be about the axial length of the flanged portion 270 or the paired flange portion 290. In some embodiments, the small amount may be equal to the axial length of the flanged portion 270 or the paired flange portion 290. Other configurations are also contemplated.

The polymeric covering 210 may be coupled to the paired flange portion 290. In some embodiments, the polymeric covering 210 may be fixedly attached to the paired flange portion 290. In some embodiments, the polymeric covering 210 may extend along an inner surface of the paired flange portion 290. In some embodiments, the polymeric covering 210 may extend along an outer surface of the paired flange portion 290. In at least some embodiments, the paired flange portion 290 may be embedded within the polymeric covering 210. Other configurations, including combinations thereof, are also contemplated.

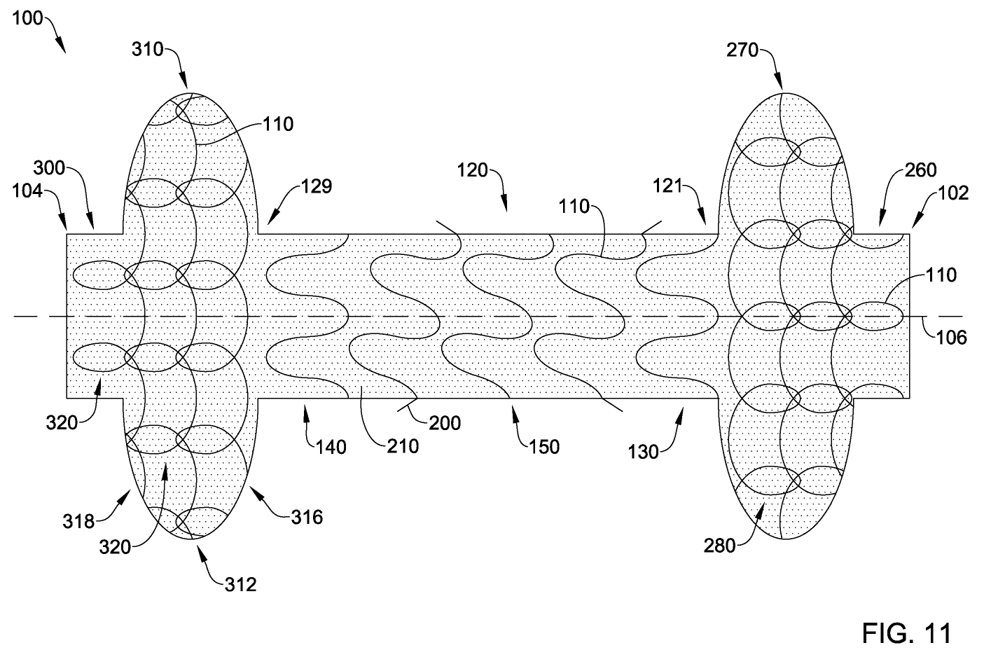

Similar to the endoprosthesis 100 described above, the endoprosthesis 100 of FIG. 11 may comprise a body portion 120 extending axially along the central longitudinal axis 106 between the second end 104 and the first end 102. The endoprosthesis 100 may comprise a first end portion 260 extending axially along the central longitudinal axis 106 from the first end 102 toward the second end 104 and/or the body portion 120. The endoprosthesis 100 may comprise a flanged portion 270 extending between the body portion 120 and the first end portion 260. The endoprosthesis 100 may comprise a second end portion 300 extending axially along the central longitudinal axis 106 from the second end 104 toward the first end 102 and/or the body portion 120. The endoprosthesis 100 may comprise a second flanged portion 310 extending between the body portion 120 and the second end portion 300. In at least some embodiments, the flanged portion 270 and the second flanged portion 310 may be axially spaced apart from each other by the body portion 120.

In some embodiments, the body portion 120, the first end portion 260, the flanged portion 270, the second end portion 300, and the second flanged portion 310 may be formed from the single wire 110 extending from the first end 102 to the second end 104. As discussed herein, the single wire 110 may form the first circumferential end segment 130, the second circumferential end segment 140, and the plurality of circumferential segments 150 extending helically around the central longitudinal axis 106 between the first circumferential end segment 130 and the second circumferential end segment 140.

In some embodiments, the second flanged portion 310 and/or the second end portion 300 may have a knitted structure, fabricated from the single wire 110 interwoven with itself, similar to the flared portion 160 and/or the flanged portion 270 described herein. In some embodiments, the second flanged portion 310 may comprise and/or the single wire 110 may form a plurality of knitted circumferential segments 320 (e.g., circumferential rows) extending circumferentially around the central longitudinal axis 106 from the body portion 120 and/or the second body end 129 to the second end portion 300 and/or the second end 104. In some embodiments, the plurality of knitted circumferential segments 320 may be constructed in a similar manner to the plurality of knitted circumferential segments 170 and/or the plurality of knitted circumferential segments 280. As such, details related to the structure and/or arrangement of the plurality of knitted circumferential segments 170 and/or the plurality of knitted circumferential segments 280 may also apply to the plurality of knitted circumferential segments 320 and are not repeated herein.

The second flanged portion 310 may comprise a medial region 312 having a medial outer diameter, wherein the medial outer diameter of the second flanged portion 310 is greater than the outer diameter of the body portion 120 and the medial outer diameter of the second flanged portion 310 is greater than an outer diameter of the second end portion 300. In some embodiments, the outer diameter of the second end portion 300 may be similar to the outer diameter of the body portion 120 and/or the outer diameter of the first end portion 260. In some embodiments, the outer diameter of the second end portion 300 may be equal to the outer diameter of the body portion 120 and/or the outer diameter of the first end portion 260. In some embodiments, the outer diameter of the second end portion 300 may be within about 10% of the outer diameter of the body portion 120 and/or the outer diameter of the first end portion 260. In some embodiments, the outer diameter of the second end portion 300 may be within about 5% of the outer diameter of the body portion 120 and/or the outer diameter of the first end portion 260. Other configurations are also contemplated.

In some embodiments, the medial outer diameter of the second flanged portion 310 may be about 20% greater than the outer diameter of the body portion 120 and/or the outer diameter of the second end portion 300. In some embodiments, the medial outer diameter of the second flanged portion 310 may be about 30% greater than the outer diameter of the body portion 120 and/or the outer diameter of the second end portion 300. In some embodiments, the medial outer diameter of the second flanged portion 310 may be about 40% greater than the outer diameter of the body portion 120 and/or the outer diameter of the second end portion 300. In some embodiments, the medial outer diameter of the second flanged portion 310 may be about 50% greater than the outer diameter of the body portion 120 and/or the outer diameter of the second end portion 300. Other configurations are also contemplated. In some embodiments, the medial outer diameter of the second flanged portion 310 may be between about 2.2 millimeters and about 45 millimeters, between about 4.8 millimeters and about 37.5 millimeters, between about 7.2 millimeters and about 30 millimeters, between about 9.6 millimeters and about 22.5 millimeters, etc. In one non-limiting example, the medial outer diameter of the second flanged portion 310 may be about 21 millimeters. Other configurations are also contemplated.

The second flanged portion 310 may comprise a first transition portion 316 extending from the body portion 120 to the medial region 312 of the second flanged portion 310. The first transition portion 316 may angle and/or curve radially outward from the body portion 120 toward and/or to the medial region 312 of the second flanged portion 310 in the deployed configuration of the endoprosthesis 100. The second flanged portion 310 may comprise a second transition portion 318 extending from the second end portion 300 to the medial region 312 of the second flanged portion 310. The second transition portion 318 may angle and/or curve radially outward from the second end portion 300 toward and/or to the medial region 312 of the second flanged portion 310 in the deployed configuration of the endoprosthesis 100.

The first transition portion 316 and the second transition portion 318 of the second flanged portion 310 may be formed from the single wire 110. In some embodiments, the first transition portion 316 may comprise at least one knitted circumferential segment of the plurality of knitted circumferential segments 320. In some embodiments, the first transition portion 316 may comprise two or more knitted circumferential segments of the plurality of knitted circumferential segments 320. In some embodiments, the second transition portion 318 may comprise at least one knitted circumferential segment of the plurality of knitted circumferential segments 320. In some embodiments, the second transition portion 318 may comprise two or more knitted circumferential segments of the plurality of knitted circumferential segments 320.

As discussed herein, at least one circumferential segment of the plurality of circumferential segments 150 may comprise an anti-migration element 200 projecting radially outward from the at least one circumferential segment of the plurality of circumferential segments 150 and/or an outer surface of the endoprosthesis 100 and/or the body portion 120. The one or more anti-migration elements and/or the anti-migration element 200 of the at least one circumferential segment of the plurality of circumferential segments 150 may be formed from the single wire 110.

Similarly, the endoprosthesis 100 may comprise a polymeric covering 210, shown in the figures via dotted shading, coupled to the body portion 120, the first end portion 260, the flanged portion 270, the second end portion 300, and the second flanged portion 310. In some embodiments, the polymeric covering 210 may be fixedly attached to the body portion 120, the first end portion 260, the flanged portion 270, the second end portion 300, and the second flanged portion 310, and/or the single wire 110, and/or the plurality of circumferential segments 150, the plurality of knitted circumferential segments 280, and the plurality of knitted circumferential segments 320. In some embodiments, the polymeric covering 210 may extend along an inner surface of the endoprosthesis 100, the single wire 110, and/or the body portion 120, the first end portion 260, the flanged portion 270, the second end portion 300, and the second flanged portion 310. In some embodiments, the polymeric covering 210 may extend along an outer surface of the endoprosthesis 100, the single wire 110, and/or the body portion 120, the first end portion 260, the flanged portion 270, the second end portion 300, and the second flanged portion 310. In at least some embodiments, the single wire 110, and/or the body portion 120, the first end portion 260, the flanged portion 270, the second end portion 300, and the second flanged portion 310 may be embedded within the polymeric covering 210. In some embodiments, the anti-migration element 200, the plurality of anti-migration elements, etc. may be covered by and/or embedded within the polymeric covering 210. Other configurations, including combinations thereof, are also contemplated.

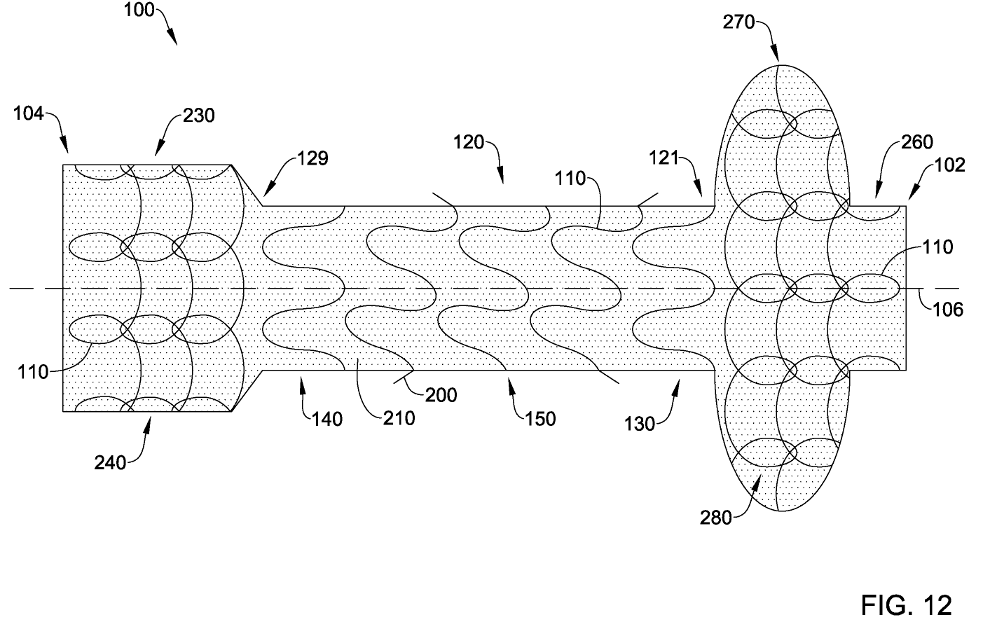

FIG. 12 schematically illustrates an example configuration of the endoprosthesis 100 combining several different elements and/or features discussed above. Other configurations and/or combinations of elements and/or features are also contemplated. The endoprosthesis 100 of FIG. 12 may comprise the elements and/or features of the endoprosthesis 100 shown and/or discussed with respect to FIG. 9 above. As such, details related to these elements and/or features are not repeated. For example, the endoprosthesis 100 may comprise the body portion 120, the first end portion 260, and the flanged portion 270 as discussed herein. Additionally, the endoprosthesis 100 may comprise the second flared portion 230 shown and discussed with respect to FIG. 8 above. Again, details related to these elements and/or features are not repeated.

The materials that can be used for the various components of the endoprosthesis and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the endoprosthesis, the single wire, the polymeric covering, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP).

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni-Co-Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An endoprosthesis extending axially along a central longitudinal axis between a first end and a second end, comprising:
a body portion; and
a flared portion extending between the body portion and the first end;
wherein the body portion and the flared portion are formed from a single wire extending from the first end to the second end;
wherein the body portion comprises an undulating arrangement of first struts and second struts defining peaks and valleys, wherein the body portion comprises:
a first circumferential end segment extending circumferentially around the central longitudinal axis at a first body end, wherein the first circumferential end segment is oriented generally perpendicular to the central longitudinal axis;
a second circumferential end segment extending circumferentially around the central longitudinal axis at a second body end, wherein the second circumferential end segment is oriented generally perpendicular to the central longitudinal axis; and
a plurality of circumferential segments extending helically around the central longitudinal axis between the first circumferential end segment and the second circumferential end segment;
wherein the flared portion comprises a plurality of knitted circumferential segments extending circumferentially around the central longitudinal axis from the body portion to the first end.

2. The endoprosthesis of claim 1, wherein the plurality of knitted circumferential segments comprises a first knitted circumferential segment disposed immediately adjacent the first circumferential end segment.

3. The endoprosthesis of claim 2, wherein the single wire comprises a transition region extending from the first circumferential end segment to the first knitted circumferential segment.

4. The endoprosthesis of claim 3, wherein the transition region extends in a generally axial direction without crossing over any portion of the single wire disposed within the first circumferential end segment.

5. The endoprosthesis of claim 3, wherein the transition region extends circumferentially and is interwoven through the first circumferential end segment.

6. The endoprosthesis of claim 3, wherein the first knitted circumferential segment of the plurality of knitted circumferential segments is interwoven with the first circumferential end segment.

7. The endoprosthesis of claim 6, wherein the first knitted circumferential segment of the plurality of knitted circumferential segments is interwoven with the transition region of the single wire.

8. The endoprosthesis of claim 1, wherein the plurality of circumferential segments is oriented at an oblique angle relative to the central longitudinal axis, as viewed in a flat pattern configuration.

9. The endoprosthesis of claim 1, further comprising a polymeric covering fixedly attached to the body portion and the flared portion.

10. The endoprosthesis of claim 1, wherein at least one circumferential segment of the plurality of circumferential segments disposed within the body portion comprises an anti-migration element projecting radially outward from the at least one circumferential segment of the plurality of circumferential segments disposed within the body portion, wherein the anti-migration element is formed from the single wire.

11. The endoprosthesis of claim 1, wherein the body portion is configured to be disposed within a first body lumen and the flared portion is configured to project into an interior space formed within a second body lumen such that the first end is spaced apart from a wall of the second body lumen.

12. The endoprosthesis of claim 11, wherein the first body lumen is a duct of a biliary system of a patient and the second body lumen is a stomach of the patient.

13. The endoprosthesis of claim 1, further comprising a second flared portion formed from the single wire and extending between the body portion and the second end.

14. The endoprosthesis of claim 13, wherein the second flared portion comprises a second plurality of knitted circumferential segments extending circumferentially around the central longitudinal axis from the body portion to the second end.

15. An endoprosthesis extending axially along a central longitudinal axis between a first end and a second end, comprising:
a body portion; and
a flared portion extending between the body portion and the first end;
wherein the body portion and the flared portion are formed from a single wire extending from the first end to the second end;
wherein the body portion comprises an undulating arrangement of first struts and second struts defining peaks and valleys, wherein the body portion comprises:
a first circumferential end segment extending circumferentially around the central longitudinal axis at a first body end, wherein centroids of the first struts and the second struts disposed within the first circumferential end segment lie within a first plane oriented generally perpendicular to the central longitudinal axis;
a second circumferential end segment extending circumferentially around the central longitudinal axis at a second body end, wherein centroids of the first struts and the second struts disposed within the second circumferential end segment lie within a second plane oriented generally perpendicular to the central longitudinal axis; and
a plurality of circumferential segments extending helically around the central longitudinal axis between the first circumferential end segment and the second circumferential end segment;
wherein the flared portion comprises a plurality of knitted circumferential segments extending circumferentially around the central longitudinal axis from the body portion to the first end.

16. The endoprosthesis of claim 15, wherein centroids of the first struts and the second struts disposed within the plurality of circumferential segments are non-coplanar.

17. The endoprosthesis of claim 16, wherein centroids of the first struts and the second struts disposed within the plurality of circumferential segments are arranged helically around the central longitudinal axis.

18. An endoprosthesis having a first end and a second end, comprising:
a body portion extending axially along a central longitudinal axis from the second end toward the first end;
a first end portion extending axially along a central longitudinal axis from the first end toward the second end; and
a flanged portion extending between the body portion and the first end portion;
wherein the body portion, the first end portion, and the flanged portion are formed from a single wire extending from the first end to the second end;
wherein the body portion comprises an undulating arrangement of first struts and second struts defining peaks and valleys, wherein the body portion comprises:
a first circumferential end segment extending circumferentially around the central longitudinal axis at a first body end, wherein the first circumferential end segment is oriented generally perpendicular to the central longitudinal axis;
a second circumferential end segment extending circumferentially around the central longitudinal axis at a second body end, wherein the second circumferential end segment is oriented generally perpendicular to the central longitudinal axis; and
a plurality of circumferential segments extending helically around the central longitudinal axis between the first circumferential end segment and the second circumferential end segment;
wherein the flanged portion comprises a plurality of knitted circumferential segments extending circumferentially around the central longitudinal axis from the body portion to the first end portion;
wherein the plurality of knitted circumferential segments extends circumferentially around the central longitudinal axis within the first end portion from the flanged portion to the first end.

19. The endoprosthesis of claim 18, further comprising a polymeric covering fixedly attached to the body portion, the first end portion, and the flanged portion.

20. The endoprosthesis of claim 19, wherein the body portion is configured to be disposed within a first body lumen and the first end portion is configured to project into an interior space formed within a second body lumen such that the first end is spaced apart from a wall of the second body lumen;
wherein the body portion is configured to span a gap between the first body lumen and the second body lumen.
